(19) United States
(12) Patent Application Publication
(10) Pub. No.: US 2020/0384084 A1
(43) Pub. Date: Dec. 10, 2020

(54) USE OF BISPECIFIC ANTIBODY AND IL-15 FOR COMBINATION THERAPY

(71) Applicant: Merus N.V., Utrecht (NL)

(72) Inventors: Alexander Berthold Hendrik BAKKER, Utrecht (NL); Pieter Fokko VAN LOO, Utrecht (NL)

(73) Assignee: Merus N.V., Utrecht (NL)

(21) Appl. No.: 16/768,447

(22) PCT Filed: Nov. 30, 2018
(86) PCT No.: PCT/NL2018/050805

§ 371 Date: May 29, 2020

Related U.S. Application Data
(60) Provisional application No. 62/593,509, filed on Dec. 1, 2017.

(51) Int. Cl. A61K 38/20; A61K 39/395; C07K 16/28

(52) U.S. Cl. CPC A61K 38/2086; A61K 39/39541; A61K 39/39558; A61K 2039/505; C07K 16/2809; C07K 2317/31; C07K 16/2851

(57) ABSTRACT

The invention relates to a method of activating a T cell in a subject and to a method of treating a cancer in a subject with a bispecific antibody and IL-15. The invention also relates to a pharmaceutical composition and to a kit comprising the bispecific antibody and IL-15. The invention further relates to a bispecific antibody for use in activating a T cell in a subject, to a bispecific antibody for use in the manufacture of a medicament for activating a T cell in a subject and to a product comprising a bispecific antibody and the mentioned IL-15.

This application claims priority to U.S. Application No. 62/593,509, filed on Dec. 1, 2017 the content of which is hereby incorporated by reference.

### FIELD OF THE INVENTION

The invention relates to a method of activating a T cell in a subject and to a method of treating a cancer in a subject with a bispecific antibody. The invention also relates to a pharmaceutical composition and to a kit comprising the bispecific antibody. The invention further relates to a bispecific antibody for use in activating a T cell in a subject, to a bispecific antibody for use in the manufacture of a medicament for activating a T cell in a subject and to a product comprising a bispecific antibody.

### BACKGROUND

Myeloid malignancies comprise a heterogeneous group of hematologic cancers characterized by expansion of clonal myeloid blasts in the bone marrow, blood, and other tissues. For example, Acute Myeloid Leukemia (AML) is an aggressive malignant disease characterized by expansion of clonal myeloid blasts in the bone marrow, blood, and other tissues. Approximately 30,000 patients are diagnosed each year with AML in Europe and US. The majority of these patients are 60 years of age or older. Although complete remission (CR) can be achieved with a number of intensive chemotherapy combinations, in 70-80% of patients younger than 60 years, for the majority of these responding patients relapse occurs with an overall 5-year survival of 40-45%. The prognosis of patients who are 60 years or older is worse, with a median survival of less than one year (Roboz, Hematology Am. Soc. Hematol. Educ. Program (2011), pp. 43-50). The current standard treatment of AML is associated with high morbidity and even mortality, and the majority of the patients in CR relapse due to remaining leukemic stem cells after chemotherapy. Further dose intensification is limited due to unacceptable toxicity.

Chronic Myeloid Leukemia (CML), also known as chronic myelogenous leukemia or chronic granulocytic leukemia, is a tri-phasic disease. It usually presents in a chronic phase (CP) marked by over-production of mature granulocytes (with <10% blasts in the blood and bone marrow), an intermediate phase which is often poorly-defined (with 10-20% blasts) and, if untreated, CML invariably transforms into an acute phase resembling acute lymphoid or myeloid leukemia (with >20% blasts in the blood or bone marrow) (Apperley, Lancet Oncol. 2007; 8(12)1116-1128). CML has a world-wide annual incidence of 1-2/100,000 population with a slight male predominance and accounts for 15% of adult leukemia (Redaelli et al, Expert Rev. Anticancer Ther. 2004; 4(1)85-96), with a median age at onset of 60 years (Hoglund et al, Ann. Hematol. 2015; 94(Suppl. 2)241-247). While a number of ABL tyrosine kinase inhibitors (TKIs) are currently approved as the front-line therapy for CML patients, TKIs fail a significant proportion of patients (Clarke et al, Exp. Hematol. 47:13-23, 2017).

Myelodysplastic syndrome (MDS) is a heterogeneous group of clonal diseases of the bone marrow, characterized by ineffective hematopoiesis leading to peripheral blood cytopenias, and in approximately 35-40% of patients, progression to acute myeloid leukemia (AML). The incidence of MDS in the general population has been reported as five new cases per 100,000 people. It affects men more frequently than it does women and, like AML and CML, its incidence increases with age (Rollison et al. Blood 112:45-52, 2008). Currently, azacitidine and decitabine have been approved as the first-line therapy for all types of MDS, but the clinical outcome of patients is poor. Moreover, allogeneic hematopoietic stem cell transplantation, which has been identified as the only potentially curative therapy for MDS patients, is useful only for a limited number of patients due to the advanced age of the majority of patients (Tefferi, N. Eng. J. Med. 361(19)1872-1875, 2009).

Accordingly, advancements of response or survival in myeloid malignancies have remained a major investigational challenge. There thus remains an urgent need for effective treatment modalities for myeloid malignancies such as AML, CML and MDS, preferably with less toxicity, especially in elderly patients.

### SUMMARY OF THE INVENTION

The present invention is based, at least in part, on the finding that administration of a CLEC12A/CD3 bispecific IgG antibody in the presence of an IL-15 moiety enhances the activation of T cells specifically targeting cells expressing levels of CLEC12A, for example, myeloid leukemia cells. This combination directs the immune system, more specifically the T cells, to kill CLEC12A expressing AML blasts and leukemic stem cells, thus providing a targeted therapy resulting in an improved prognosis for cancer patients than currently approved therapies.

In one aspect, provided is a method of activating a T cell in a subject, the method comprising administering to the subject a CLEC12A/CD3 bispecific antibody and an IL-15 moiety.

In a related aspect, provided is a method of treating a cancer in a subject, the method comprising administering to the subject a CLEC12A/CD3 bispecific antibody and an IL-15 moiety.

Also provided are a pharmaceutical composition and a kit comprising a CLEC12A/CD3 bispecific antibody and an IL-15 moiety formulated in single formulation or separate formulations.

In addition are provided:
a CLEC12A/CD3 bispecific antibody for use in activating a T cell in a subject, wherein the CLEC12A/CD3 bispecific antibody is administered simultaneously, separately or sequentially with an IL-15 moiety;
a CLEC12A/CD3 bispecific antibody for use in the manufacture of a medicament for activating a T cell in a subject, wherein the CLEC12A/CD3 bispecific antibody is administered simultaneously, separately or sequentially with an IL-15 moiety;
a product comprising a CLEC12A/CD3 bispecific antibody and an IL-15 moiety as a combined preparation for simultaneous, separate or sequential use in activating a T cell in a subject; and
a CLEC12A/CD3 bispecific antibody and an IL-15 moiety for use in the treatment of a cancer in a subject.

Other features and advantages of the instant disclosure will be apparent from the following detailed description and examples, which should not be construed as limiting.

### BRIEF DESCRIPTION OF THE FIGURES

FIG. 1 is a bar graph which depicts the results of a cytotoxicity assay with healthy donor T cells, co-cultured with HL-60 cells at an E:T ratio of 5:1 in the presence of IgGs tested as indicated.

FIG. 2A is a graph, which shows the effect of IL-15 on PB9122p01 induced CD4+ T cell activation in a HL-60 cytotoxicity assay with healthy donor T cells co-cultured with HL-60 cells at an E:T ratio of 1:10 for 72 hours. CD4+ T cell activation was quantified by flow cytometry.

FIG. 2B is a graph which shows the effect of IL-15 on PB9122p01 induced CD8+ T cell activation in a HL-60 cytotoxicity assay with healthy donor T cells co-cultured with HL-60 cells at an E:T ratio of 1:10 for 72 hours. CD8+ T cell activation was quantified by flow cytometry.

### DETAILED DESCRIPTION

In one aspect, provided is a method of activating a T cell in a subject, comprising administering to the subject a CLEC12A/CD3 bispecific antibody and an IL-15 moiety. In some embodiments, the method activates a T cell that specifically engages a cell expressing CLEC12A. In a related aspect, there is provided a method of treating a cancer in a subject, the method comprising administering to the subject a CLEC12A/CD3 bispecific antibody and an IL-15 moiety. In some embodiments, the cancer is one which expresses CLEC12A.

An IL-15 moiety enhances the activation of T cells targeted to cells expressing levels of CLEC12A, for example of myeloid leukemia cells, in the presence of a CLEC12A/CD3 bispecific IgG antibody. An IL-15 moiety supports the survival of T cells that may be present at a low frequency, for example in the case of AML. This combination directs the immune system, more specifically the T cells, to kill CLEC12A expressing AML blasts and leukemic stem cells, thus providing a targeted therapy resulting in an improved prognosis for cancer patients than currently approved therapies.

In the method of the invention, administering the CLEC12A/CD3 bispecific antibody and the IL-5 moiety may activate the T cell to specifically target a CLEC12A expressing cell. Administering the CLEC12A/CD3 bispecific antibody and the IL-5 moiety may activate the T cell to specifically target and lyse a CLEC12A expressing cell.

As disclosed herein, the CLEC12A/CD3 bispecific antibody and the IL-15 moiety can be administered concurrently or in either order. For example, the CLEC12A/CD3 bispecific antibody and the IL-15 moiety may be administered simultaneously, separately or sequentially. In some embodiments, administration of a CLEC12A/CD3 bispecific antibody is carried out before administration of an IL-15 moiety. In other embodiments, administration of an IL-15 moiety is carried out before administration of a CLEC12A/CD3 bispecific antibody. In other embodiments, both a CLEC12A/CD3 bispecific antibody and an IL-15 moiety are administered simultaneously.

In order that the present description may be more readily understood, certain terms are first defined. Additional definitions are set forth throughout the detailed description. Unless stated otherwise, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents unless the context clearly dictates otherwise. Use of the term “including” as well as other forms, such as “include”, “includes”, and “included”, is not limiting.

As used herein, the term “CLEC12A” refers to C-type lectin domain family 12 member A. CLEC12A is also referred to as C-Type Lectin Protein CLL-1; MICL; Dendritic Cell-Associated Lectin 2; C-Type Lectin Superfamily; Myeloid Inhibitory C-Type Lectin-Like Receptor; C-Type Lectin-Like Molecule-1; DCAL2; CLL1; C-Type Lectin-Like Molecule 1; DCAL-2; Killer cell lectin like receptor subfamily L, member 1 (KLRL1); CD371 (cluster of differentiation 371) (Bakker A. et al. Cancer Res. 2004, 64, p 8843 50; GenBank™ access. no: AY547296; Zhang W. et al. GenBank™ access. no: AF247788; A. S. Marshall, et al. J Biol Chem 2004, 279, p 14792-802; GenBank™ access. no: AY498550; Y. Han et al. Blood 2004, 104, p 2858 66; H. Floyd, et al. GenBank™ access. no: AY426759; C. H. Chen, et al. Blood 2006, 107, p 1459 67). Ids: HGNC: 31713; Entrez Gene: 160364; Ensembl: ENSG00000172322; OMIM: 612088; UniProtKB: Q5QGZ9.

CLEC12A is an antigen that is expressed on leukemic blast cells and on leukemic stem cells in acute myeloid leukemia (AML), including the CD34 negative or CD34 low expressing leukemic stem cells (side population) (A. B. Bakker et al. Cancer Res 2004, 64, p 8443 50; Van Rhenen et al. 2007 Blood 110:2659; Moshaver et al. 2008 Stem Cells 26:3059), as well as in myelodysplastic syndromes (MDS) (Bakker et al. 2004, supra and Toff-Peterson et al., Br. J. Haematol. 175(3)393-401, 2016). Expression of CLEC12A is otherwise thought to be restricted to cells of the hematopoietic lineage, particularly to myeloid lineage in peripheral blood and bone marrow, i.e., granulocytes, monocytes and dendritic cell precursors. More importantly, CLEC12A is absent on normal hematopoietic stem cells. Where reference is made to CLEC12A herein, the reference is to human CLEC12A (SEQ ID NO: 1), unless specifically stated otherwise.

The term “CLEC12A” means all variants (such as splice and mutation) that are referenced herein and isoforms thereof that retain the myeloid expression profile (both at surface expression level and mRNA level) including as described in Bakker et al. Cancer Res 2004, 64, p 8443-50 and Marshall 2004—J Biol Chem 279(15), p 14792-802. While accession numbers are primarily provided as a further method of identification, the actual sequence of the protein may vary, for instance because of a mutation in the encoding gene such as those occurring in some cancers or the like.

The term “CD3” (cluster of differentiation 3) refers a protein complex, which is composed of a CD3γ chain (SwissProt P09693), a CD3δ chain (SwissProt P04234), CD3ε chains (SwissProt P07766), and a CD3 zeta chain homodimer (SwissProt P20963). CD3ε is known under various aliases some of which are: “CD3e Molecule, Epsilon (CD3-TCR Complex)”; “CD3e Antigen, Epsilon Polypeptide (TiT3 Complex)”; T-Cell Surface Antigen T3/Leu-4 Epsilon Chain; T3E; T-Cell Antigen Receptor Complex, Epsilon Subunit Of T3; CD3e Antigen; CD3-Epsilon 3; IMD18; TCRE. Ids for CD3E Gene are HGNC: 1674; Entrez Gene: 916; Ensembl: ENSG00000198851; OMIM: 186830 and UniProtKB: P07766. These chains associate with the T-cell receptor (TCR) and the ζ-chain to generate an activation signal in T lymphocytes. The TCR, ζ-chain, and CD3 molecules together comprise the TCR complex. CD3 is expressed on T cells. Where reference is made to CD3 herein, the reference is to human CD3 (SEQ ID NOs: 2-5), unless specifically stated otherwise.

The terms “IL-15” and “IL-15 moiety” are used interchangeably herein to refer to a peptide or protein moiety having a biological activity of IL-15. An IL-15 or IL-15 moiety may have a biological activity of a native IL-15, typically native human IL-15, as well as any protein or polypeptide substantially homologous thereto. The term includes naturally, recombinantly and synthetically produced moieties, as well as peptides and proteins modified, for example, by mutation. These terms also include analogs having additional glycosylation sites, analogs having at least one or more additional amino acids at the carboxy terminal end and IL-15 fragments.

The term “IL-15 fragment” means any protein or polypeptide having the amino acid sequence of a portion or fragment of an IL-15 moiety and which has the biological activity of IL-15 (and is thus a functional fragment). Fragments include proteins or polypeptides produced by proteolytic degradation of an IL-15 moiety as well as proteins or polypeptides produced by chemical synthesis by methods routine in the art.

As used herein, the terms “native IL-15” and “native interleukin-15” refer to any naturally occurring mammalian interleukin-15 amino acid sequences, including immature or precursor and mature forms. Interleukin-15 (IL-15) is a member of the four alpha-helix bundle family of lymphokines produced by many cells in the body. Native IL-15 plays a pivotal role in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity. Non-limiting examples of GeneBank Accession Nos. for the amino acid sequence of various species of native mammalian interleukin-15 include NP000576 (human, immature form), CAA62616 (human, immature form), NP—001009207 (Felis catus, immature form), AAB94536 (rattus, immature form), AAB41697 (rattus, immature form), NP—032383 (Mus musculus, immature form), AAR19080 (canine), AAB60398 (macaca mulatta, immature form), AAI00964 (human, immature form), AAH23698 (Mus musculus, immature form), and AAH18149 (human). Where herein reference is made to native IL-15, the reference is to native human IL-15 (SEQ ID NO: 6), unless specifically stated otherwise.

The IL-15 receptor consists of three polypeptides, the type-specific IL-15 receptor alpha (“IL-15Ra”), the IL-2/IL-15 receptor-β (or CD122), and the common γ chain (or CD132) that is shared by multiple cytokine receptors. IL-15 signaling has been shown to occur through the heterodimeric complex of IL-15Ra, β and γ, through the heterodimeric complex of β and γ, or through a subunit, IL-15RX, found on mast cells. An IL-15 receptor may be a “native IL-15Ra” and “native interleukin-15 receptor alpha” which, in the context of proteins or polypeptides, refers to any naturally occurring mammalian interleukin-15 receptor alpha (“IL-15Ra”) amino acid sequence, including immature or precursor and mature forms and naturally occurring isoforms. Non-limiting examples of GeneBank Accession Nos. for the amino acid sequence of various native mammalian IL-15Ra include NP.sub.—002180 (human), ABK41438 (Macaca mulatta), NP.sub.—032384 (Mus musculus), Q60819 (Mus musculus), CA141082 (human). Where herein reference is made to IL-15Ra, the reference is to mature human IL-15Ra (SEQ ID NO: 7), unless specifically stated otherwise, for example, soluble human IL-15Ra (SEQ ID NO: 8).

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units—each with two heavy chains and two light chains. Antibodies for therapeutic use are preferably as close to natural antibodies of the subject to be treated as possible (for instance human antibodies for human subjects). An antibody according to the present invention is not limited to any particular format or method of producing it.

A “bispecific antibody” is an antibody as described herein wherein one domain of the antibody binds to a first antigen whereas a second domain of the antibody binds to a second antigen, wherein said first and second antigens are not identical. The term “bispecific antibody” also encompasses antibodies wherein one heavy chain variable region/light chain variable region (VH/VL) combination binds a first epitope on an antigen and a second VH/VL combination that binds a second epitope. The term further includes antibodies wherein a VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. The light chain of the present invention can also be a light chain as specified herein, having from 0 to 10, preferably from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen or epitope binding specificity of the IgG.

“Percent (%) identity” as referred to amino acid sequences herein is defined as the percentage of amino acid residues in a candidate sequence that are identical with the amino acid residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this invention the NEEDLE program from the EMBOSS package may be used to determine percent identity of amino acid and nucleic acid sequences (version 2.8.0 or higher, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment.

As an antibody typically recognizes an epitope of an antigen, and such an epitope may be present in other compounds as well, antibodies according to the present invention that “specifically recognize” an antigen, for example, CLEC12A or CD3, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation. Methods of determining spatial conformation of epitopes are known to persons of ordinary skill in the art and include techniques in the art for example, x-ray crystallography, HDX-MS and 2-dimensional nuclear magnetic resonance, pepscan, and alanine scan depending on the nature of the epitope (see, e.g., Epitope Mapping Protocols in Methods in Molecular Biology, Vol. 66, G. E. Morris, Ed. (1996)).

The term ‘aberrant cells’ as used herein includes tumor cells, more specifically tumor cells of hematological origin including also pre-leukemic cells such as cells that cause myelodysplastic syndromes (MDS) and leukemic cells such as acute myeloid leukemia (AML) tumor cells or chronic myelogenous leukemia (CML) cells.

The term ‘immune effector cell’ or ‘effector cell’ as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, and including cells of the myeloid lineage, such as monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte. The recruitment of effector cells to aberrant cells means that immune effector cells are brought in proximity to the aberrant target cells such that the effector cells can directly kill, or indirectly initiate the killing of the aberrant cells.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, reduce the number of cancer cells, decrease tumor size or number of tumor cells, decrease the presence of circulating tumor cells, reduce or prevent metastases of a tumor, slow or arrest tumor growth and/or prevent or delay tumor recurrence or relapse.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of a CLEC12A/CD3 bispecific antibody and an IL-15 moiety, in combination, to effect a decrease in a cancer (for example a decrease in the number of cancer cells) or slowing of progression of a cancer, such as acute myeloid leukemia, myelodysplastic syndrome or chronic myelogenous leukemia. An effective amount of the combination therapy is administered according to the methods described herein in an “effective regimen” which refers to a combination of the CLEC12A/CD3 bispecific antibody and an IL-15 moiety, wherein the order of administration and dosage frequency is adequate to effect treatment.

As used herein, the terms “synergy”, “therapeutic synergy”, and “synergistic effect” refer to a phenomenon where treatment of patients with a combination of therapeutic agents (e.g., CLEC12A/CD3 bispecific antibody in combination with an IL-15 moiety) manifests a therapeutically superior outcome to the outcome achieved by each individual constituent of the combination when used alone (see, e.g., T. H. Corbett et al., 1982, Cancer Treatment Reports, 66, 1187). In this context a therapeutically superior outcome includes one or more of the following (a) an increase in therapeutic response that is greater than the sum of the separate effects of each agent alone at the same dose as in the combination; (b) a decrease in the dose of one or more agents in the combination without a decrease in therapeutic efficacy; (c) a decrease in the incidence of adverse events while receiving a therapeutic benefit that is equal to or greater than the monotherapy of each agent at the same dose as in the combination, (d) a reduction in dose-limiting toxicities while receiving a therapeutic benefit that is greater than the monotherapy of each agent; (e) a delay or minimization of the induction of drug resistance. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone. Synergism of a drug combination may be determined, for example, according to the combination index (CI) theorem of Chou-Talalay (Chou et al., Adv. Enzyme Regul. 1984; 22:27-55; Chou, Cancer Res. 2010; 70(2)440-446).

“Relapse” or “recurrence” or “resurgence” are used interchangeably herein, and refer to the radiographic diagnosis of return, or signs and symptoms of return of cancer after a period of improvement or response.

### II. CLEC12A/CD3 Bispecific Antibodies

As disclosed herein bispecific antibodies for use in the methods provided herein include bispecific antibodies that comprise one heavy chain variable region/light chain variable region (VH/VL) combination that binds CLEC12A, and a second VH/VL combination that binds CD3 thereof.

### Antigen Binding Regions

Suitable CLEC12A heavy chain variable (VH) regions for use in a CLEC12A/CD3 bispecific antibody include those which bind to CLEC12A. In preferred embodiments, the CLEC12A VH region of a CLEC12A/CD3 bispecific antibody binds to CLEC12A expressed on tumor cells. Exemplary CLEC12A VH regions for use in the CLEC12A/CD3 bispecific antibody are disclosed, for example, in WO2017/010874, WO2014/051433, and WO2005/000894 (each of which is incorporated herein by reference).

In some embodiments, the binding affinity of the CLEC12A/CD3 bispecific antibody for CLEC12A on tumor cells is at least 2 times, 4 times, 6 times, 10 times, 20 times, 30 times, 40 times or 50 times higher than the affinity of binding to CD3. In certain embodiments, the CLEC12A binding affinity of the CLEC12A/CD3 bispecific antibody for CLEC12A is between about 1×10⁻⁶ M and 1×10⁻¹⁰ M, between about 1×10⁻⁷M and 1×10⁻¹⁰ M, or between about 1×10⁻⁸ and 1×10⁻¹°. In some embodiments, CLEC12A binding affinity of the CLEC12A/CD3 bispecific antibody for CLEC12A is at least 1×10⁻⁸ M, preferably at least 1×10⁻⁹ M. In certain embodiments, the binding affinity of the CLEC12A/CD3 bispecific antibody for CLEC12A is between about 1×10⁻⁸ M and 1×10⁻⁹ M including, for example, about 2×10⁻⁹ M, about 3×10⁻⁹ M, about 4×10⁻⁹ M, about 5×10⁻⁹ M, about 6×10⁻⁹ M, about 7×10⁻⁹ M, 8×10⁻⁹ M or about 9×10⁻⁹ M, and the binding affinity of the CLEC12A/CD3 bispecific antibody for CD3 is at least 30 times lower, at least 40 times lower, or at least 50 times lower.

In certain embodiments, the CD3 VH region of the CLEC12A/CD3 bispecific antibody binds to cell surface expressed CD3/TCR on human T cell lines with an affinity (K_{D}) that is significantly less than the affinity of murine anti-CD3 antibody, mOKT3. In some embodiments, the CD3 VH region of the the CLEC12A/CD3 bispecific antibody binds to CD3 with a binding affinity of at least 1×10⁻⁶ M. In some embodiments, the CD3 binding affinity of the bispecific antibody is between about 1×10⁻⁶ M and 1×10⁻¹⁰ M. In some embodiments, the CD3 binding affinity of the the CD3 VH region of the bispecific antibody is between about 1×10⁻⁷M-1×10⁻⁸ M.

In some embodiments, the CLEC12A/CD3 bispecific antibody comprises a first heavy chain variable region that binds human CLEC12A, wherein the heavy chain variable region comprises:
(a) a heavy chain CDR1 comprising the amino acid sequence SGYTFTGY (SEQ ID NO: 9), a heavy chain CDR2 comprising the amino acid sequence IINPSGGS (SEQ ID NO: 10), and a heavy chain CDR3 comprising the amino acid sequence GTTGDWFDY (SEQ ID NO: 11);
(b) a heavy chain CDR1 comprising the amino acid sequence SGYTFTSY (SEQ ID NO: 13), a heavy chain CDR2 comprising the amino acid sequence IINPSGGS (SEQ ID NO: 14), and a heavy chain CDR3 comprising the amino acid sequence GNYGDEFDY (SEQ ID NO:15); or
(c) a heavy chain CDR1 comprises the amino acid sequence SGYTFTGY (SEQ ID NO: 17), a heavy chain CDR2 comprising the amino acid sequence WINPNSGG (SEQ ID NO: 18), and a heavy chain CDR3 comprising the amino acid sequence DGYFADAFDY (SEQ ID NO:.19).

Conservative variations of 1, 2 or 3 amino acid residues from the recited CDR sequences are allowed while retaining the same kind of binding activity (in kind, not necessarily in amount). Hence, said heavy chain CDR 1, 2 and 3 sequences preferably contain sequences that deviate in no more than three, preferably no more than two, more preferably no more than one amino acid from the recited CDR sequences. In certain embodiments, the heavy chain CDR 1, 2 and 3 sequences are identical to the recited CDR sequences.

In some embodiments, the CLEC12A/CD3 bispecific antibody comprises a heavy chain variable region that binds human CLEC12A, wherein said heavy chain variable region comprises the HCDR1, HCDR2 and HCDR3 of the VH region set forth in SEQ ID NOs: 12, 16 or 20.

In some embodiments, the CLEC12A/CD3 bispecific antibody comprises a heavy chain variable region that binds human CLEC12A, wherein said heavy chain variable region comprises an amino acid sequence at least 90%, preferably at least 95%, more preferably at least 97%, more preferably at least 98%, more preferably at least 99% identical or 100% identical to the amino acid sequence set forth in SEQ ID NOs: 12, 16 or 20.

In some embodiments, the heavy chain variable region of the bispecific antibody that binds human CLEC12A can have 0-10, preferably 0-5 amino acid insertions, deletions, substitutions, additions in the sequence of the heavy chain variable region outside of the three CDR sequences, or a combination thereof. In some embodiments, the heavy chain variable region comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain embodiments, the CLEC12A/CD3 bispecific antibody comprises a heavy chain variable region that binds human CLEC12A, wherein said heavy chain variable region comprises an amino acid sequence selected from SEQ ID NOs: 12, 16 and 20.

CD3 heavy chain variable (VH) regions suitable for use in the CLEC12A/CD3 bispecific antibody include those which can bind a CD3γ chain, a CD3δ chain, a CD3ε chain or a combination of CD3δ/CD3ε or CD3γ/CD3ε. In some embodiments, the CD3 VH region of the bispecific antibody binds the CD3ε chain. In some embodiments, the CD3 VH region binds to human CD3. In some embodiments, the CD3 VH region binds to the human CD3ε chain.

Exemplary CD3 binding regions for use in the CLEC12A/CD3 bispecific antibody are disclosed in WO2017/010874, WO2014/051433 and WO2005/118635 (each of which is incorporated herein by reference).

In certain embodiments, the CLEC12A/CD3 bispecific antibody comprises a heavy chain variable region that binds CD3, wherein said heavy chain variable region comprises:
(a) a heavy chain CDR1 comprising the amino acid sequence GFTFSSYG (SEQ ID NO: 21), a heavy chain CDR2 sequence comprising the amino acid sequence IWYNGRKQ (SEQ ID NO: 22), and a heavy chain CDR3 comprising the amino acid sequence GTGYNWFDP (SEQ ID NO: 23);
(b) a heavy chain CDR1 comprising the amino acid sequence GFTFSSYG (SEQ ID NO: 21), a heavy chain CDR2 sequence comprising the amino acid sequence IWYSGSKKN(SEQ ID NO: 30), and a heavy chain CDR3 comprising the amino acid sequence GTGYNWFDP (SEQ ID NO: 23);
(c) a heavy chain CDR1 comprising the amino acid sequence GFTFSSYG (SEQ ID NO: 21), a heavy chain CDR2 sequence comprising the amino acid sequence IWYHGRKQ (SEQ ID NO: 32), and a heavy chain CDR3 comprising the amino acid sequence GTGYNWFDP (SEQ ID NO: 23);
(d) a heavy chain CDR1 comprising the amino acid sequence GFTFSSYG (SEQ ID NO: 21), a heavy chain CDR2 sequence comprising the amino acid sequence IWYHARKQ (SEQ ID NO: 34), and a heavy chain CDR3 comprising the amino acid sequence GTGYNWFDP (SEQ ID NO: 23);
(e) a heavy chain CDR1 comprising the amino acid sequence GFTFSSYG (SEQ ID NO: 21), a heavy chain CDR2 sequence comprising the amino acid sequence IWYNARKQ (SEQ ID NO: 36), and a heavy chain CDR3 comprising the amino acid sequence GTGYNWFDP (SEQ ID NO: 23);
(g) a heavy chain CDR1 comprising the amino acid sequence GFTFSSYG (SEQ ID NO: 21), a heavy chain CDR2 sequence comprising the amino acid sequence IWYNTRKQ (SEQ ID NO: 45), and a heavy chain CDR3 comprising the amino acid sequence GTGYNWFDP (SEQ ID NO: 23);
(h) a heavy chain CDR1 comprising the amino acid sequence GFTFSSYG (SEQ ID NO: 21), a heavy chain CDR2 sequence comprising the amino acid sequence IWYDGKNT (SEQ ID NO: 47), and a heavy chain CDR3 comprising the amino acid sequence GTGYNWFDP (SEQ ID NO: 23);
(i) a heavy chain CDR1 comprising the amino acid sequence GFTFSGYG (SEQ ID NO: 21), a heavy chain CDR2 sequence comprising the amino acid sequence IYYDGSRT (SEQ ID NO: 49), and a heavy chain CDR3 comprising the amino acid sequence GTGYNWFDP (SEQ ID NO: 23); or
(j) a heavy chain CDR1 comprising the amino acid sequence GFTFSKYG (SEQ ID NO: 21), a heavy chain CDR2 sequence comprising the amino acid sequence IWHDGRKT (SEQ ID NO: 51), and a heavy chain CDR3 comprising the amino acid sequence GTGYNWFDP (SEQ ID NO: 23).

The CDR1 comprising the amino acid sequence GFTFSSYG (SEQ ID NO: 21) is defined according to IMGT. The CDR1 may comprise the amino acid sequence SYGMH (SEQ ID NO: 60) as defined according to Kabat.

Variations of 1, 2 or 3 amino acid residues from the recited CDR sequences are allowed while retaining the same kind of binding activity (in kind, not necessarily in amount). Hence, said heavy chain CDR 1, 2 and 3 sequences preferably contain sequences that deviate in no more than three, preferably no more than two, more preferably no more than one amino acid from the recited CDR sequences. In certain embodiments, the heavy chain CDR 1, 2 and 3 sequences are identical to the recited CDR sequences.

In some embodiments, the CLEC12A/CD3 bispecific antibody comprises a heavy chain variable region that binds human CD3, wherein said heavy chain variable region comprises the HCDR1, HCDR2 and HCDR3 of the VH region set forth in SEQ ID NOs: 24-29, 31, 33, 35, 37-44, 46, 48, 50 and 52.

In some embodiments, the CLEC12A/CD3 bispecific antibody comprises a heavy chain variable region that binds human CD3, wherein said heavy chain variable region comprises at least 90%, preferably at least 95%, more preferably at least 97%, more preferably at least 98%, more preferably at least 99% identical or 100% identical to the amino acid sequence of one of the VH region sequences set forth in SEQ ID NO: 24-29, 31, 33, 35, 37-44, 46, 48, 50 and 52.

In certain embodiments, the CLEC12A/CD3 bispecific antibody comprises a heavy chain variable region that binds human CD3, wherein said heavy chain variable region comprises at least 90%, preferably at least 95%, more preferably at least 97%, more preferably at least 98%, more preferably at least 99% identical or 100% identical to the amino acid sequence of one of the sequences VH region set forth in SEQ ID NO: 37-44.

In some embodiments, the heavy chain variable region of the bispecific antibody that binds human CD3 can have from 0 to 10, preferably from 0 to 5 amino acid insertions, deletions, substitutions, additions in the sequence of the heavy chain variable region outside of the three CDR sequences, or a combination thereof. In some embodiments, the heavy chain variable region comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

Additional variants of the disclosed amino acid sequences which retain CLEC12A or CD3 binding can be obtained, for example, from phage display libraries which contain the rearranged human IGKV1-39/IGKJ1 VL region (De Kruif et al. Biotechnol Bioeng. 2010 (106)741-50), and a collection of VH regions incorporating amino acid substitutions into the amino acid sequence of a CLEC12A or CD3 VH region disclosed herein, as previously described (e.g., US 2016/0368988). Phages encoding Fab regions which bind CLEC12A or CD3 may be selected and analyzed by flow cytometry, and sequenced to identify variants with amino acid substitutions, insertions, deletions or additions which retain antigen binding. For example, as described in US 2016/0368988, the CD3 VH region may be substituted at position A50 and be modified by an S, Y, M or a Q; D59 may be substituted by L, I, V, F, R, A, N, H, S, T, Y or E, preferably by an Y or an E; A61 may be substituted by N, I, H, Q, L, R, Y, E, S, T, D, K, V; and F105 may be substituted by an Y or an M. Tolerated amino acid substitutions can readily be found using the method described in example 5A together with CIEX-HPLC after storage.

In certain embodiments, the CLEC12A/CD3 bispecific antibody comprises a heavy chain variable region that binds human CD3, wherein said heavy chain variable region comprises an amino acid sequence selected from SEQ ID NO: 24-29, 31, 33, 35, 37-44, 46, 48, 50 or 52.

In certain embodiments, CLEC12A/CD3 bispecific antibody comprises a heavy chain variable region that binds human CD3, wherein said heavy chain variable region comprises an amino acid sequence selected from SEQ ID NO: 37-44.

In certain embodiments, the CLEC12A/CD3 bispecific antibody comprises a first heavy chain variable region that binds human CLEC12A, wherein the first VH region comprises an amino acid sequence that is at least 90%, preferably at least 95%, more preferably at least 97%, more preferably at least 98%, more preferably at least 99% identical or 100% identical to the amino acid sequence of the VH region set forth in SEQ ID NO: 12, 16 and 20; and a second heavy chain variable region that binds human CD3 comprising, wherein the second VH region comprises an amino acid sequence that is at least 90%, preferably at least 95%, more preferably at least 97%, more preferably at least 98%, more preferably at least 99% identical or 100% identical to the amino acid sequence of one of the VH region sequences set forth in SEQ ID NO: 37-44.

In certain embodiments, the CLEC12A/CD3 bispecific antibody comprises a first heavy chain variable region that binds human CLEC12A, and a second heavy chain variable region that binds human CD3, wherein
(a) the first heavy chain variable region comprises:
   (i) a heavy chain CDR1 comprising the amino acid sequence SGYTFTGY (SEQ ID NO: 9), a heavy chain CDR2 comprising the amino acid sequence IINPSGGS (SEQ ID NO: 10), and a heavy chain CDR3 comprising the amino acid sequence GTTGDWFDY (SEQ ID NO: 11);
   (ii) a heavy chain CDR1 comprising the amino acid sequence SGYTFTSY (SEQ ID NO: 13), a heavy chain CDR2 comprising the amino acid sequence IINPSGGS (SEQ ID NO: 14), and a heavy chain CDR3 comprising the amino acid sequence GNYGDEFDY (SEQ ID NO: 15); or
   (iii) a heavy chain CDR1 comprises the amino acid sequence SGYTFTGY (SEQ ID NO: 17), a heavy chain CDR2 comprising the amino acid sequence WINPNSGG (SEQ ID NO: 18), and a heavy chain CDR3 comprising the amino acid sequence DGYFADAFDY (SEQ ID NO: 19); and
(b) the second heavy chain variable region comprises,
   (i) a heavy chain CDR1 comprising the amino acid sequence GFTFSSYG (SEQ ID NO: 21), a heavy chain CDR2 sequence comprising the amino acid sequence IWYNGRKQ (SEQ ID NO: 22), and a heavy chain CDR3 comprising the amino acid sequence GTGYNWFDP (SEQ ID NO: 23);
   (ii) a heavy chain CDR1 comprising the amino acid sequence GFTFSSYG (SEQ ID NO: 21), a heavy chain CDR2 sequence comprising the amino acid sequence IWYSGSKKN (SEQ ID NO: 30), and a heavy chain CDR3 comprising the amino acid sequence GTGYNWFDP (SEQ ID NO: 23);
   (iii) a heavy chain CDR1 comprising the amino acid sequence GFTFSSYG (SEQ ID NO: 21), a heavy chain CDR2 sequence comprising the amino acid sequence IWYHGRKQ (SEQ ID NO: 32), and a heavy chain CDR3 comprising the amino acid sequence GTGYNWFDP (SEQ ID NO: 23);
   (iv) a heavy chain CDR1 comprising the amino acid sequence GFTFSSYG (SEQ ID NO: 21), a heavy chain CDR2 sequence comprising the amino acid sequence IWYHARKQ (SEQ ID NO: 34), and a heavy chain CDR3 comprising the amino acid sequence GTGYNWFDP (SEQ ID NO: 23);
   (v) a heavy chain CDR1 comprising the amino acid sequence GFTFSSYG (SEQ ID NO: 21), a heavy chain CDR2 sequence comprising the amino acid sequence IWYNARKQ (SEQ ID NO: 36), and a heavy chain CDR3 comprising the amino acid sequence GTGYNWFDP (SEQ ID NO: 23);
   (vi) a heavy chain CDR1 comprising the amino acid sequence GFTFSSYG (SEQ ID NO: 21), a heavy chain CDR2 sequence comprising the amino acid sequence IWYNTRKQ (SEQ ID NO: 45), and a heavy chain CDR3 comprising the amino acid sequence GTGYNWFDP (SEQ ID NO: 23);
   (vii) a heavy chain CDR1 comprising the amino acid sequence GFTFSSYG (SEQ ID NO: 21), a heavy chain CDR2 sequence comprising the amino acid sequence IWYDGKNT (SEQ ID NO: 47), and a heavy chain CDR3 comprising the amino acid sequence GTGYNWFDP (SEQ ID NO: 23);
   (viii) a heavy chain CDR1 comprising the amino acid sequence GFTFSGYG (SEQ ID NO: 21), a heavy chain CDR2 sequence comprising the amino acid sequence IYYDGSRT (SEQ ID NO: 49), and a heavy chain CDR3 comprising the amino acid sequence GTGYNWFDP (SEQ ID NO: 23); or
   (ix) a heavy chain CDR1 comprising the amino acid sequence GFTFSKYG (SEQ ID NO: 21), a heavy chain CDR2 sequence comprising the amino acid sequence IWHDGRKT (SEQ ID NO: 51), and a heavy chain CDR3 comprising the amino acid sequence GTGYNWFDP (SEQ ID NO: 23).

In certain embodiments, the CLEC12A/CD3 bispecific antibody comprises a first heavy chain variable region that binds human CLEC12A, wherein the first VH region comprises a heavy chain CDR1 comprising the amino acid sequence SGYTFTGY (SEQ ID NO: 9), a heavy chain CDR2 comprising the amino acid sequence IINPSGGS (SEQ ID NO: 10), and a heavy chain CDR3 comprising the amino acid sequence GTTGDWFDY (SEQ ID NO: 11); and a second heavy chain variable region, wherein the second VH region comprises a heavy chain CDR1 comprising the amino acid sequence GFTFSSYG (SEQ ID NO: 21), a heavy chain CDR2 comprising the amino acid sequence IWYNARKQ (SEQ ID NO: 36), and a heavy chain CDR3 comprising the amino acid sequence GTGYNWFDP (SEQ ID NO: 23).

In certain embodiments, the CLEC12A/CD3 bispecific antibody comprises a first heavy chain variable region that binds human CLEC12A, wherein the amino acid sequence of the first VH region is selected from SEQ ID NOs: 12, 16 and 20; and a second heavy chain variable region that binds human CD3, wherein the amino acid sequence of the second VH region is selected from SEQ ID NOs: 24-29, 31, 33, 35, 37-44, 46, 48, 50 and 52.

In certain embodiments, the CLEC12A/CD3 bispecific antibody comprises a first heavy chain variable region that binds human CLEC12A, wherein the amino acid sequence of the first VH region is SEQ ID NO: 12; and a second heavy chain variable region that binds human CD3, wherein the amino acid sequence of the second VH region is selected from SEQ ID NOs: 37-44.

In one embodiment, the CLEC12A/CD3 bispecific antibody comprises a first heavy chain variable region that binds human CLEC12A, wherein the amino acid sequence of the first VH region is set forth in SEQ ID NO: 12; and a second heavy chain variable region that binds human, wherein the amino acid sequence of the second VH region is set forth in SEQ ID NO: 37.

### Light Chain Variable Regions

The light chain variable regions of the VH/VL CLEC12A binding region and the VH/VL binding region of the CD3 binding region of the CLEC12A/CD3 bispecific antibody may be the same as the VL region of parental CLEC12A monospecific antibody and/or the VL region of parental CD3 monospecific antibody, or alternative VL regions may be used for one or both VH/VL region combinations as long as the bispecific antibody retains binding to both the CLEC12A and CD3 antigens.

In some embodiments, the VL region of the VH/VL CLEC12A binding region of the CLEC12A/CD3 bispecific antibody is similar to the VL region of the VH/VL CD3 binding region. In certain embodiments, VL regions in the first and second VH/VL region combinations are identical.

In certain embodiments, the light chain variable region of one or both VH/VL binding regions of the CLEC12A/CD3 bispecific antibody comprises a common light chain variable region. In some embodiments, the common light chain variable region of one or both VH/VL binding regions comprises a germline 012 variable region V-segment. In certain embodiment, the light chain variable region of one or both VH/VL binding regions comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39; 012a or 012. External Ids for the gene are HGNC: 5740; Entrez Gene: 28930; Ensembl: ENSG00000242371. The amino acid sequence for the V-region is provided in SEQ ID NO: 56. The V-region can be combined with one of five J-regions. Preferred J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain embodiments, the light chain variable region of one or both VH/VL binding regions comprises the kappa light chain IgVκ1-39*01/IGJκ1*01 or IgVκ1-39*01/IGJκ1*05 (SEQ ID NO: 57 and SEQ ID NO: 58, respectively).

In some embodiments, the light chain variable region of one or both VH/VL binding regions of the CLEC12A/CD3 bispecific antibody comprises an LCDR1 comprising the amino acid sequence QSISSY (SEQ ID NO: 53), an LCDR2 comprising the amino acid sequence AAS, and an LCDR3 comprising the amino acid sequence QQSYSTP (SEQ ID NO: 55) (i.e., the CDRs of IGKV1-39 according to IMGT). In some embodiments, the light chain variable region of one or both VH/VL binding regions of the CLEC12A/CD3 bispecific antibody comprises an LCDR1 comprising the amino acid sequence QSISSY (SEQ ID NO: 53), an LCDR2 comprising the amino acid sequence AASLQS (SEQ ID NO: 54), and an LCDR3 comprising the amino acid sequence QQSYSTP (SEQ ID NO: 55)

In some embodiments, one or both VH/VL binding regions of the CLEC12A/CD3 bispecific antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in SEQ ID NO: 57. In some embodiments, one or both VH/VL binding regions of the CLEC12A/CD3 bispecific antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in SEQ ID NO: 58.

For example, in some embodiments, the variable light chain of one or both VH/VL binding regions of the CLEC12A/CD3 bispecific antibody can have from 0 to 10, preferably from 0 to 5 amino acid insertions, deletions, substitutions, additions or a combination thereof with respect to SEQ ID NO: 57 or SEQ ID NO: 58. In some embodiments, the light chain variable region of one or both VH/VL binding regions of the CLEC12A/CD3 bispecific antibody comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other embodiments, the light chain variable region of one or both VH/VL binding regions of the CLEC12A/CD3 bispecific antibody comprises the amino acid sequence of SEQ ID NO: 57 or SEQ ID NO: 58. In certain embodiments, both VH/VL binding regions of the CLEC12A/CD3 bispecific antibody comprise identical VL regions. In one embodiment, the VL of both VH/VL binding regions of the CLEC12A/CD3 bispecific antibody comprises the amino acid sequence set forth in SEQ ID NO: 57. In one embodiment, the VL of both VH/VL binding regions of the CLEC12A/CD3 bispecific antibody comprises the amino acid sequence set forth in SEQ ID NO: 58.

### Bispecific Formats

CLEC12A/CD3 bispecific antibodies for use in the methods disclosed herein can be provided in a number of formats. Many different formats of bispecific antibodies are known in the art, and have been reviewed by Kontermann (Drug Discov Today, 2015 July; 20(7):838-47; MAbs, 2012 March-April; 4(2):182-97) and in Spiess et al., (Alternative molecular formats and therapeutic applications for bispecific antibodies. Mol. Immunol. (2015) http://dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In some embodiments, the CLEC12A/CD3 bispecific antibodies used in the methods provided herein are generally of the human IgG subclass (e.g., for instance IgG1, IgG2, IgG3, IgG4). In certain embodiments, the antibodies are of the human IgG1 subclass. Full length IgG antibodies are preferred because of their favorable half-life and for reasons of low immunogenicity. Accordingly, in certain embodiments, the CLEC12A/CD3 bispecific antibody is a full length IgG molecule. In an embodiment, the CLEC12A/CD3 bispecific antibody is a full length IgG1 molecule.

Accordingly, in certain embodiments, the CLEC12A/CD3 bispecific antibody comprises a fragment crystallizable (Fc). The Fc of the CLEC12A/CD3 bispecific antibody is preferably comprised of a human constant region. A constant region or Fc of the CLEC12A/CD3 bispecific antibody may contain one or more, preferably not more than 10, preferably not more than 5 amino-acid differences with a constant region of a naturally occurring human antibody. For example, in certain embodiments, each Fab-arm of the bispecific antibodies may further include an Fc-region comprising modifications promoting the formation of the bispecific antibody, modifications affecting Fc-mediated effector functions, and/or other features described herein.

In preferred embodiments, a CLEC12A/CD3 bispecific full length IgG antibody has a mutated lower hinge and/or CH2 domains such that interaction of said bispecific IgG antibody with Fc gamma (Fcγ) receptors is reduced. As used herein, the term “such that interaction of said bispecific IgG antibody with Fc gamma receptors is reduced” means that the interaction of the CLEC12A/CD3 bispecific antibody with Fc gamma receptors, if such Fc gamma receptors are present in the vicinity of the antibody, is reduced. In certain embodiments, the interaction of the CLEC12A/CD3 bispecific antibody with the Fc receptor is essentially abolished. Bispecific antibodies with reduced Fcγ receptor binding have been previously described (US 2014/0120096, incorporated herein by reference).

In certain embodiments, the CLEC12A/CD3 bispecific antibody comprises a mutated lower hinge and/or CH2 domain with at least one substitution at amino acids positions 235 and/or 236 (EU numbering). Preferably, both amino acids positions 235 and 236 are substituted. As described in US 2014/0120096, substitutions at these sites are capable of essentially preventing the interaction between an antibody and the Fc receptor present on tumor cells or effector cells. Accordingly, in certain embodiments, the CLEC12A/CD3 bispecific antibody comprises a mutated CH2 and/or lower hinge domains comprising an L235G and/or G236R substitution. Preferably, both L235G and G236R are substituted. Alternatively, a person skilled in the art may introduce lower hinge and/or the CH2 domain mutations that comprise the substitutions 234F, 235E and/or 331S (Oganesyan et al. Biol. Crystall. 2008(D64)700). Preferably, all three substitutions are introduced in this alternative.

### Bispecific Antibody Production and Isolation

Bispecific antibodies are typically produced by cells that express nucleic acid(s) encoding the antibody. Accordingly, in some embodiments, the bispecific CLEC12A/CD3 antibodies disclosed herein are produced by providing a cell comprising one or more nucleic acids that encode the heavy and light chain variable regions and constant regions of the bispecific CLEC12A/CD3 antibody. The cell is preferably an animal cell, more preferably a mammal cell, more preferably a primate cell, most preferably a human cell. A suitable cell is any cell capable of comprising and preferably of producing the CLEC12A/CD3 bispecific antibody.

Suitable cells for antibody production are known in the art and include a hybridoma cell, a Chinese hamster ovary (CHO) cell, an NS0 cell or a PER-C6 cell. Various institutions and companies have developed cell lines for the large scale production of antibodies, for instance for clinical use. Non-limiting examples of such cell lines are CHO cells, NS0 cells or PER.C6 cells. In a particularly preferred embodiment said cell is a human cell. Preferably a cell that is transformed by an adenovirus E1 region or a functional equivalent thereof. A preferred example of such a cell line is the PER.C6 cell line or equivalent thereof. In a particularly preferred embodiment said cell is a CHO cell or a variant thereof. Preferably a variant that makes use of a Glutamine synthetase (GS) vector system for expression of an antibody. In one preferred embodiment, the cell is a CHO cell.

In some embodiments, the cell expresses the different light and heavy chains that make up the CLEC12A/CD3 bispecific antibody. In certain embodiments, the cell expresses two different heavy chains and at least one light chain. In one preferred embodiment, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1-39/IGKJ1 (huVκ1-39) light chain. The huVκ1-39 was previously shown to be able to pair with more than one heavy chain thereby giving rise to antibodies with diverse specificities, which facilitates the generation of bispecific molecules (De Kruif et al. J. Mol. Biol. 2009 (387) 548-58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred embodiment the bispecific antibody of the invention comprises two different immunoglobulin heavy chains with compatible heterodimerization domains. Various compatible heterodimerization domains have been described in the art.

The compatible heterodimerization domains are preferably compatible immunoglobulin heavy chain CH3 heterodimerization domains. The art describes various ways in which such hetero-dimerization of heavy chains can be achieved.

One preferred method for producing the CLEC12A/CD3 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in one embodiment the heavy chain/light chain combination that comprises the variable domain that binds CLEC12A, comprises a DE variant of the heavy chain. In this embodiment the heavy chain/light chain combination that comprises the variable domain that binds CD3 comprises a KK variant of the heavy chain.

### CLEC12A/CD3 Bispecific Antibody Characterization

A candidate CLEC12A/CD3 IgG bispecific antibody can be tested for binding using any suitable assay. For example, binding to membrane-expressed CD3 on HPB-ALL cells can be assessed by flow cytometry (according to the FACS procedure as previously described in WO2014/051433). In one embodiment, the binding of a candidate CLEC12A/CD3 bispecific antibody to CD3 on HPB-ALL cells is demonstrated by flow cytometry, performed according to standard procedures known in the art. Binding to cell-expressed CD3 is confirmed using CHO cell transfected with CD3δ/ε or CD3γ/ε. The binding of the candidate bispecific IgG1 to CLEC12A is determined using CHO cells transfected with a CLEC12A expression construct; a CD3 monospecific antibody and a CLEC12A monospecific antibody, as well as an irrelevant IgG1 isotype control mAb are included in the assay as controls (e.g., an antibody which binds CD3 and another antigen such as tetanus toxin (TT)).

The affinities of the CD3 and CLEC12A Fabs of a candidate CLEC12A/CD3 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human CLEC12A (Sino Biological Inc, cat. Nr. 11896-H07H) and human CD3δε-Fc protein are run over the sensor surface in a concentration range to measure on- and off-rates. After each cycle, the sensor surface is regenerated by a pulse of HCl and the bsAb is captured again. From the obtained sensorgrams, on- and off-rates and affinity values for binding to human CD3 and CLEC12A are determined using the BIAevaluation software, as previously described (e.g., US 2016/0368988).

The T-cell stimulatory capacity of a CLEC12A/CD3 bispecific antibody can be determined in an assay using healthy donor resting T-cells obtained according to the procedure described in WO2014/051433 and US 2016/0368988. For example, a candidate CLEC12A/CD3 bispecific antibody is tested in purified healthy donor resting T-cells incubated with cells from the leukemia-derived HL60 cell line in 10% fetal bovine serum (FBS) or 10% normal human serum (HS) at an effector: target cell ratio of 10:1 or 5:1 for two days. Results are expressed as the percentage of CD69-positive or CD25-positive cells within the CD4-positive or CD8-positive T-cell population.

The ability of a CLEC12A/CD3 bispecific antibody to induce target cell lysis may be tested in an assay using leukemia cells, e.g., HL-60 cells, labeled with carboxyfluorescein diacetate succimidyl ester (CFSE) and co-cultured with T-cells from healthy donor, according to the procedure described in WO2014/051433 and US 2016/0368988. The surviving CFSE-positive cells are quantified by flow cytometry, and the results are expressed as the percentage of specific lysis related to the PBS control condition.

### III. IL-15 Moieties

Any molecule that binds an IL-15 receptor and activates the receptor (e.g., acts as an IL-15 receptor agonist) can be used in the present methods. Suitable IL-15 receptor agonists are known in the art and include naturally-occurring IL-15; recombinant IL-15, synthetic IL-15; modified IL-15; PEGylated IL-15; fusion proteins comprising IL-15 and a heterologous fusion partner; and functional fragments of naturally-occurring IL-15 that bind to and activate an IL-15R; and IL-15 mimetics. The IL-15 can produced in any convenient manner, including isolation of naturally-occurring IL-15 from human, mouse, rat, etc. tissues; production by synthetic means; and production by recombinant means. IL-15 amino acid sequences are known in the art. See, e.g., GenBank Accession Nos. NP751915, NP751914, NP000576, NP037261, NP032383, and P97604.

Exemplary IL-15 moieties are described herein, in the literature, and in, for example, US 2006/0104945, US 2015/0359853, US 2017/0020963, WO 2017/062835, Pettit et al. (1997 J. Biol. Chem. 272(4)2312-2318), and Wong et al. (2013 OncoImmunology 2(11)e26442: 1-3). Multimeric IL-15 soluble fusion molecules, e.g., including complexes of an IL-15 superagonist mutant and a dimeric IL-15Ra domains (IL-15RaSu/Fc) (U.S. Pat. Nos. 9,255,141 and 9,328,159); and recombinant human IL-15 (rhIL-15) is commercially available (e.g. (Peprotech, Rocky Hill, N.J.; CellGenix).

In some embodiments, the IL-15 moiety is recombinant human IL-15 (hIL-15). In other embodiments, the IL-15 moiety is a soluble IL-15 receptor or receptor fragment (sIL-15Ra). In other embodiments, the IL-15 moiety is a complex comprising IL-15 and sIL-15Ra, for example, as described in U.S. Pat. Nos. 9,255,141 and 9,328,159. In still other embodiments, the IL-15 moiety is a long-acting form of IL-15, for example, a conjugate of IL-15 and a water soluble polymer as described, for example, in WO 2015/815373.

In some embodiments, the IL-15 moieties can include means known in the art to extend bioavailability, including PEG, mPEG, dextran, PVP, PVA, polyamino acids such as poly-L-lysine or polyhistidine, albumin and gelatin at specific sites on the IL-15 molecule that can interfere with binding of IL-15 to the β or γ subunits of the IL-15 receptor complex, while maintaining the high affinity of IL-15 for the IL-15R subunit. Additionally, IL-15 can be specifically glycosylated at sites that can interfere with binding of IL-15 to the β or γ subunits of the IL-15 receptor complex, while maintaining the high affinity of IL-15 for the IL-15R subunit. Preferred groups for conjugation are PEG, dextran, and PVP. The PEG moieties can be bonded to IL-15 at known sites to take advantage of PEG's large molecular size and can be bonded to IL-15 by utilizing lysine or cysteine residues naturally occurring in the protein or by site-specific PEGylation.

To produce additional IL-15 moieties, the sequence of any known IL-15 polypeptide may be altered in various ways known in the art to generate targeted changes in sequence. A variant polypeptide will usually be substantially similar to the naturally occurring sequence, i.e., will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine). Techniques for substituting and adding amino acid residues and non-naturally occurring amino acid residues are well known to those of ordinary skill in the art (e.g., J. March, Advanced Organic Chemistry: Reactions Mechanisms and Structure, 4th Ed. (New York: Wiley-Interscience, 1992).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to modification with a polyethylene glycol moiety (PEGylation); and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine. For example, an IL-15 polypeptide may comprise an N-blocked species, wherein the N-terminal amino acid is acylated with an acyl group, such as a formyl group, an acetyl group, a malonyl group, and the like. Also suitable for use is a consensus IL-15, e.g., an IL-15 that comprises an amino acid sequence that is a consensus of two or more known IL-15 amino acid sequences.

Also suitable for use are IL-15 polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability. The polypeptides may be fused to albumin or another heterologous fusion partner.

Truncated versions, hybrid variants, and peptide mimetics of IL-15 can also serve as the IL-15 moiety. Biologically active fragments, deletion variants, substitution variants or addition variants of any of the foregoing that maintain at least some degree of IL-15 activity can also serve as an IL-15 moiety.

An IL-15 moiety may be prepared by in vitro synthesis, using conventional methods as known in the art, by recombinant methods, or may be isolated from cells induced or naturally producing the protein. If desired, various groups may be introduced into the polypeptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like. For example, IL-15 polypeptides may be produced by expression of DNA sequences transformed or transfected into bacterial hosts, e.g., E. coli, or in eukaryotic host cells (e.g., yeast; mammalian cells, such as CHO cells; and the like). In these embodiments, the IL-15 is “recombinant IL-15.” Where the host cell is a bacterial host cell, the IL-15 may be modified to comprise an N-terminal methionine.

Various methods for determining in vitro IL-15 activity are described in the art. For example, the activity of IL-15 can be assessed in a pSTAT assay. Briefly, if an IL-15-dependent CTLL-2 cell is exposed to a test article having IL-15 activity, initiation of a signaling cascade results that includes the phosphorylation of STATS at tyrosine residue 694 (Tyr694) that can be quantitatively measured. Assay protocols and kits are known and include, for example, the MSD Phospho(Tyr694)/Total STATa,b Whole Cell Lysate Kit (Meso Seal Diagnostics, LLC, Gaithersburg, Md.). A suitable IL-15 moiety for use in the methods provided herein may exhibit a pSTAT5 EC50 value of less than about 300 ng/mL (more preferably less than about 150 ng/mL) for at least one of 5 minutes or 10 minutes.

### IV. Pharmaceutical Compositions

In one aspect, provided is a pharmaceutical composition comprising a CLEC12A/CD3 bispecific antibody, an IL-15 moiety and a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra-articularly and the like) the active compound may be coated in a material to protect the compound from the action of acids and other natural conditions that may inactivate the compound.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage.

Also included are solid preparations which are intended for conversion, shortly before use, to liquid preparations for either oral or parenteral administration. Such liquid forms include solutions, suspensions and emulsions.

### V. Treatment

The compositions and methods provided herein are particularly useful for activating a T cell or T cells in a patient. The patient may be suffering from a cancer. Accordingly, the compositions and methods may be used in the treatment of various myeloid malignancies, including myeloid leukemias and pre-leukemic diseases of myeloid origin. Accordingly, disease that can be treated according to the methods provided herein include myeloid leukemias (e.g., AML and CML) or pre-leukemic diseases such myelodysplastic syndrome (MDS) (and progression to AML).

As used herein, combined administration (co-administration) includes simultaneous administration of the CLEC12A/CD3 bispecific antibody and IL-15 moiety in the same or different dosage form, separate administration or sequential administration. Accordingly, in some embodiments, a CLEC12A/CD3 bispecific antibody may be used in a method for activating a T cell in a subject, wherein the CLEC12A/CD3 bispecific antibody is administered simultaneously, separately or sequentially with an IL-15 moiety. In other embodiments, a CLEC12A/CD3 bispecific antibody may be used in the treatment of a cancer in a subject, wherein the CLEC12A/CD3 bispecific antibody may be administered simultaneously, separately or sequentially with an IL-15 moiety.

In other embodiments, a CLEC12A/CD3 bispecific antibody may be for use in the manufacture of a medicament for activating a T cell in a subject, wherein the CLEC12A/CD3 bispecific antibody is administered simultaneously, separately or sequentially with an IL-15 moiety. A product comprising a CLEC12A/CD3 bispecific antibody and an IL-15 moiety may be a combined preparation for simultaneous, separate or sequential use in activating a T cell in a subject. In other embodiments, a CLEC12A/CD3 bispecific antibody may be for use in the manufacture of a medicament for treating a cancer in a subject, wherein the CLEC12A/CD3 bispecific antibody may be administered simultaneously, separately or sequentially with an IL-15 moiety. A product comprising a CLEC12A/CD3 bispecific antibody and an IL-15 moiety may be a combined preparation for simultaneous, separate or sequential use in treating a cancer in a subject.

The CLEC12A/CD3 bispecific antibody and IL-15 moiety can be administered according to a suitable dosage, route (e.g., intravenous, intraperitoneal, intramuscular, intrathecal or subcutaneous).

The CLEC12A/CD3 bispecific antibody and IL-15 moiety can also be administered according to any suitable schedule. For example, the CLEC12A/CD3 bispecific antibody and IL-15 moiety can be simultaneously administered in a single formulation. Alternatively, the CLEC12A/CD3 bispecific antibody and IL-15 moiety can be formulated for separate administration, wherein they are administered concurrently or sequentially.

For example, in some embodiments, the CLEC12A/CD3 bispecific antibody can be administered first followed by the administration of the IL-15 moiety, or vice versa. Dosage regimens in the above methods of treatment and uses are adjusted to provide the optimum desired response (e.g., a therapeutic response). The actual dose provided in the methods will vary depend upon the age, weight, and general condition of the subject as well as the severity of the condition being treated and the judgment of the health care professional.

For example, a single bolus may be administered, several divided doses may be administered over time or the dose may be proportionally reduced or increased as indicated by the exigencies of the therapeutic situation. In one embodiment, the CLEC12A/CD3 bispecific antibody is administered prior to administration of the IL-15 moiety, e.g., the CLEC12A/CD3 bispecific antibody is administered into the patient first, followed from later by an administration of the IL-15 moiety. In one embodiment, the IL-15 moiety is administered prior to administration of the CLEC12A/CD3 bispecific antibody, e.g., the IL-15 moiety is administered into the patient first, followed from later by administration of the CLEC12A/CD3 bispecific antibody (e.g., one or more minutes, hours or days after). Such concurrent or sequential administration results in both the CLEC12A/CD3 bispecific antibody and IL-15 moiety being simultaneously present in treated patients. Concurrent presence of both the CLEC12A/CD3 bispecific antibody and IL-15 moiety will support CLEC12A/CD3 bispecific antibody induced T cell function and CLEC12A/CD3 bispecific antibody mediated T cell-induced target cell lysis.

In another embodiment, the IL-15 moiety and the CLEC12A/CD3 bispecific antibody are administered simultaneously.

In one embodiment, a subject is administered a single dose of an IL-15 moiety and a single dose of the CLEC12A/CD3 bispecific antibody. In some embodiments, the CLEC12A/CD3 bispecific antibody and IL-15 moiety will be administered repeatedly, over a course of treatment. For example, in certain embodiments, multiple (e.g., 2, 3, 4, 5, 6, 7, 8, 9, 10 or more) doses of an IL-15 moiety and multiple (e.g., 2, 3, 4, 5, 6, 7, 8, 9, 10 or more) doses of a CLEC12A/CD3 bispecific antibody are administered to a subject in need of treatment.

In some embodiments, administrations of a IL-15 moiety and a CLEC12A/CD3 bispecific antibody may be done weekly or monthly, in which regimen, they may be administered on the same day (e.g., simultaneously), or one after the other (e.g., one or more minutes, hours or days before or after one another). When administered separately, the CLEC12A/CD3 bispecific antibody and IL-15 moiety may be, but are not necessarily administered according to the same administration (i.e., dosing) protocol. For example, one cycle of treatment may comprise administering the CLEC12A/CD3 bispecific antibody one or multiple times, while a therapeutically effective dose of the IL-15 moiety may be administered either more or less frequently the CLEC12A/CD3 bispecific antibody. In certain embodiments, administration of each dose of the IL-15 moiety and the CLEC12A/CD3 bispecific antibody may be on the same day, or alternatively, the IL-15 moiety may be administered 1 or more days before or after the CLEC12A/CD3 antibody.

In some embodiments, the dose of the CLEC12A/CD3 bispecific antibody and/or IL-15 moiety is varied over time. For example, the CLEC12A/CD3 bispecific antibody and/or IL-15 moiety may be initially administered at a high dose and may be lowered over time. In another embodiment, the CLEC12A/CD3 bispecific antibody and/or IL-15 moiety is initially administered at a low dose and increased over time.

In another embodiment, the amount of the CLEC12A/CD3 bispecific antibody and/or IL-15 moiety are administered is constant for each dose. In another embodiment, the amount of the CLEC12A/CD3 bispecific antibody and/or IL-15 moiety varies with each dose. For example, the maintenance (or follow-on) dose of each can be higher or the same as the loading dose which is first administered. In another embodiment, the maintenance dose of the each can be lower or the same as the loading dose. A clinician may utilize preferred dosages as warranted by the condition of the patient being treated. The dose may depend upon a number of factors, including stage of disease, etc. The specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain embodiments, the CLEC12A/CD3 bispecific antibody is administered at a dose of 0.1, 0.3, 1, 2, 3, 4, 5, 6, 7, 8, 9, or 10 mg/kg body weight. In another embodiment, the CLEC12A/CD3 bispecific antibody is administered at a dose of 0.5, 1, 2, 3, 4, 5, 6, 7, 8, 9, or 10 mg/kg body weight.

In some embodiments, the IL-15 moiety used in the methods provided herein is recombinant human IL-15 (rhIL-15). In certain embodiments, the rhIL-15 is administered at doses of about 0.125 μg/kg per day to 2.0 μg/kg per day. In certain embodiments, the rhIL-15 is administered at 0.125, 0.25, 0.5, 1.0 or 2.0 μg/kg per day. In certain embodiments, the rhIL-15 is administered by intravenous bolus. In other embodiments, the rhIL-15 is administered by continuous intravenous infusion (CIV). In certain embodiments, the rhIL-15 is administered by CIV for between 2 to 10 days, 5 to 10 days, or 7 to 10 days. In one embodiment, the rhIL-15 is administered by CIV for 10 days. In related embodiments, the rhIL-15 is administered in treatment cycle of between about 30 and 60 days, in which the rhIL-15 is administered by CIV for the first 5 to 10 days of each cycle.

In some embodiment, the IL-15 moiety administered according to the methods provided herein is a soluble IL-15 receptor or receptor fragment (sIL-15Ra). In other embodiments, the IL-15 moiety is a complex comprising IL-15 and sIL-15Ra, for example, as described in U.S. Pat. Nos. 9,255,141 and 9,328,159. In certain embodiments, the IL-15/IL-15Ra complex is administered at a dose of approximately 0.1 μg/kg to approximately 20 μg/kg, approximately 10 μg/kg to approximately 20 μg/kg, approximately 20 μg/kg to approximately 40 μg/kg, or approximately 25 μg/kg to 50 μg/kg to a subject.

In certain embodiments, the IL-15 moiety is an IL-15/IL-15Ra which is administered subcutaneously. In some embodiments, the IL-15/IL-15Ra complex is administered at a frequency of every day, every other day, every 3, 4, 5, 6 or 7 days. In certain embodiments, the IL-15/IL-15Ra is administered 1, 2, 3, 4, 5, 6 or 7 days per week. In certain embodiments, the dose of the first cycle and each subsequent cycle is 0.1 μg/kg to 1 μg/kg, 1 μg/kg to 5 μg/kg, or 5 μg/kg to 10 μg/kg. In another embodiment, the first dose of the first cycle and each subsequent cycle is 0.1 μg/kg to 0.5 μg/kg, 1 μg/kg to 2 μg/kg, 1 μg/kg to 3 μg/kg, 2 μg/kg to 5 μg/kg, or 2 μg/kg to 4 μg/kg. In another embodiment, the dose of the first cycle and each subsequent cycle is 0.1 μg/kg, 0.25 μg/kg, 0.5 μg/kg, 1 μg/kg, 1.25 μg/kg, 1.5 μg/kg, 1.75 μg/kg, 2 μg/kg, 2.25 μg/kg, 2.5 μg/kg, 2.75 μg/kg, 3 μg/kg, 3.25 μg/kg, 3.5 μg/kg, 4 μg/kg, 4.25 μg/kg, 4.5 μg/kg, 4.75 μg/kg, or 5 μg/kg. In one embodiment, the IL-15/IL-15Ra complex is administered according to a 28 day cycle in which the IL-15/IL-15Ra complex is administered subcutaneously three time per week for two consecutive weeks.

In still other embodiments, the IL-15 moiety used in the methods provided herein is a long-acting form of IL-15, for example, a conjugate of IL-15 and a water soluble polymer as described, for example, in WO 2015815373. One of ordinary skill in the art can determine the quantity of a long-acting, IL-15 agonist that is sufficient to provide clinically relevant agonist activity at the IL-15 receptor (“IL-15R”). In some embodiments, the IL-15 polymer conjugate is administered at a dose of from about 0.001 mg/kg to about 10 mg/kg, preferably from about 0.001 to about 5 mg/kg. In certain embodiments, the IL-15 polymer conjugate is administered at a dose of about 0.03 mg/kg to about 3 mg/kg. In certain embodiments, the IL-15 polymer conjugate is administered at a dose of about 0.03 mg/kg, about 0.1 mg/kg, about 0.3 mg/kg or about 3.0 mg/kg. In other embodiments, the IL-15 polymer conjugate is administered at a dose of about 0.0025 mg/kg, about 0.008 mg/kg, about 0.01 mg/kg, about 0.025 mg/kg, 0.05 mg/kg or about 0.25 mg/kg.

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable); increased number of NK (natural killer) cells; increased number of CLEC12A specific T cells; and increased number of CLEC12A specific memory T cells.

With regard to the frequency of administering the CLEC12A/CD3 bispecific antibody, one of ordinary skill in the art will be able to determine an appropriate frequency. For example, a clinician can decide to administer the CLEC12A/CD3 bispecific antibody relatively infrequently (e.g., once every two weeks) and progressively shorten the period between doses as tolerated by the patient. With regard to frequency of administering the IL-15 moiety, the frequency for these agents can be determined in a similar fashion. Exemplary lengths of time associated with the course of therapy in accordance with the claimed method include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

### Outcomes

The efficacy of the treatment methods provided herein can be assessed using any suitable means. In one embodiment, the clinical efficacy of the combination treatment is analyzed using AML blast reduction in the bone marrow as an objective response criterion. Patients, e.g., humans, treated according to the methods disclosed herein preferably experience improvement in at least one sign of cancer. In some embodiments, one or more of the following can occur: the number of cancer cells can be reduced; cancer recurrence is prevented or delayed; one or more of the symptoms associated with cancer can be relieved to some extent. In addition, in vitro assays to determine the T-cell mediated target cell lysis (as described in WO2017/010874) can be performed with AML tumor blasts isolated from the subject.

In another embodiment, the methods of treatment produce a comparable clinical benefit rate (CBR=CR (complete response), PR (partial response) or SD (stable disease)≥6 months) better than that achieved by a CLEC12A/CD3 bispecific antibody or an IL-15 moiety (e.g., rhIL-15) alone.

In some embodiment, the tumor cells are no longer detectable following treatment as described herein. In some embodiments, a subject is in partial or full remission. In certain embodiments, a subject has an increased overall survival, median survival rate, and/or progression free survival.

### VI. Additional Agents/Therapies

The combinations of the present invention (e.g., CLEC12A/CD3 bispecific antibody in combination with IL-15 moiety) may also be used in conjunction with other well-known therapies that are selected for their particular usefulness against the cancer that is being treated. Combinations of the instant invention may alternatively be used sequentially with known pharmaceutically acceptable agent(s) when appropriate.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### VII. Kits and Articles of Manufacture

Also provided herein is a kit or a product which includes a pharmaceutical composition containing a CLEC12A/CD3 bispecific antibody and an IL-15 moiety, and a pharmaceutically-acceptable carrier, in a therapeutically effective amount adapted for use in the preceding methods. In some embodiments, the kit or product optionally also can include instructions, e.g., comprising administration schedules, to allow a practitioner (e.g., a physician, nurse, or patient) to administer the composition contained therein to a patient having cancer.

In some embodiments, the kit or product include multiple packages of the single-dose pharmaceutical compositions each containing an effective amount of a CLEC12A/CD3 bispecific antibody and an IL-15 moiety for a single administration in accordance with the methods provided above. Instruments or devices necessary for administering the pharmaceutical composition(s) also may be included in the kit or product. For instance, a kit or product may provide one or more pre-filled syringes containing a unit dosage of a CLEC12A/CD3 bispecific antibody and an IL-15 moiety in the same container, or in separate containers to be administered as separate and distinct compositions.

In certain embodiments, one or both of a CLEC12A/CD3 bispecific antibody and an IL-15 moiety is provided in a solid form suitable for reconstitution and subsequent administration in accordance with the accompanying instructions.

In yet further embodiments, the composition or combination or kit or product includes one or more additional active agents.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate embodiments, however, it will be appreciated that the scope of the invention may include embodiments having combinations of all or some of the features described.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Example 1

### Effect of IL-15 on CLEC12A Specific HL60 Cytotoxicity Induced by CLEC12A/CD3 Bispecific IgG

### Materials and Methods

### Reagents

1. IMDM (Invitrogen, #21980-065)
2. PBS (Braun, #220/12257974/1110)
3. HS serum (PAA, #C15-020 lot nr 13)
4. FBS
5. Facs buffer PBS/0.5% BSA
6. BSA (Sigma, #A99647)
7. HL-60 cell line (Culture should be in log phase)
8. Healthy donor derived heparinized blood
9. Pan T cell isolation kit (Miltenyi biotec, #130-096-535)
10. CFSE (Life Technology, #C11-57)
11. 96 well flat bottom plate (Costar #3596)
12. IL-15 (stock conc.20 ug/ml, Miltenyi #130-095-766)

### Antibodies for FACS

1. CD3PeCY7 (UCHT1)(Biolegend, #300420)
2. CD25PE (Beckman Coulter, #A07774)
3. CD69APC (Beckman Coulter, #A80711)
4. CD8PECY7 (Beckman Coulter #737661)
5. CD4ECD (Beckman Coulter #6604727)

### Antibodies and Dilutions Made

| | | Fab specificity | Fab specificity | Code # | Code # | | Conc. |
|---|---|---|---|---|---|---|---|
| | Antibody | MF1 | MF2 | MF1 | MF2 | Fc-tail* | Included |
| 1 | PBS | — | — | — | — | — | — |
| 2 | PG1207p17 | TT | NA | 1207 | — | WT | 1000 ng/ml |
| 3 | PG5196p02 | CD3 | NA | 5196 | — | WT | 1000 ng/ml |
| 4 | PB9122p01 | CLEC12A | CD3 | 4327 | 5196 | DM-DEKK | 1000 ng/ml |
| 5 | PB9124p01 | TT | CD3 | 1337 | 5196 | DM-DEKK | 1000 ng/ml |

* WT = wildtype; DM = Fc comprising L235G and G236R; DEKK = Fc comprising L351D/L368E and L351KT366K.

Isolation of CD3+ T Cells from Peripheral Blood Mononuclear Cells

PBMC were isolated from heparinized blood collected from a healthy donor, washed and suspended in MACS buffer and a 50 μl sample was taken for counting and rest centrifuged to pellet the cells (5 min 500G, RT). Cells were re-suspended in MACS buffer at a density of 10×10⁶/40 μl MACS buffer and the CD3 selection protocol provided by the manufacturer (http://www.miltenyibiotec.com/download/datasheets_en/1897/DS130_096_535.pdf).

The number and purity of the purified CD3+ T cells was determined by counting chamber and FACS, respectively. For FACS, 100 μl of the purified CD3+ T cells were incubated with 1 μl CD3PECY7 for 20 min at 4° C. and measured on FACS machine. For counting, isolated T cells were frozen in 10% DMSO containing medium and stored in liquid nitrogen until use. For the assay, CD3+ T cells were thawed at 37° C. in the water bath, washed using excess assay medium, centrifuged (5 min, 500G, RT), and re-suspended at a density of 40,000 cells/ml. 50 μl of above cells were used to provide a density per well to get 2000 CD3+ T cells/assay well.

### CFSE Labeling of HL-60 Parental Cell Line

The HL-60 cell line was grown in suspension according to previously described methods, and harvested from the culture flask. Samples were taken to determine cell density and for use as a non-CFSE labeled control.

The cells were washed (5 min 500G, RT) once with excess PBS, and resuspended at a density of 10×10⁶/ml in PBS. A 2000-fold dilution of CFSE stock (conc. 5 mM) in PBS to a concentration of 2.5 μM was utilized. Labeling was carried out by adding one volume of HL-60 (density 10×10⁶/ml in PBS) and one volume of 2.5 μM CFSE in PBS, gently mixing followed by incubation for 10 min at 37° C. An equal volume of FBS was added and the mix incubated at RT for 2 min. The labeled cells were pelleted by centrifugation (5 min 500G, RT) and the supernatant discarded. The cells were resuspended and washed twice in excess medium with serum used for cytoxicity assay. The cells were then resuspended in serum containing the assay medium (approximately 1×10⁶/ml). Samples were used to count (10 μl) and to check the CFSE labeling efficiency (100 μl), and the cell density was adjusted to 0.2×10⁶/ml using assay medium.

### Results

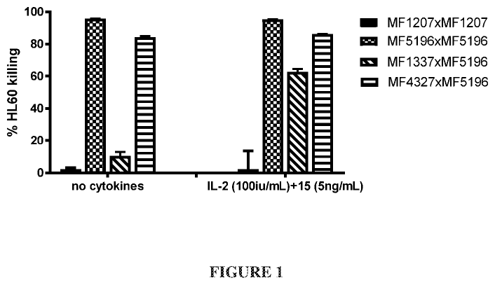

To enhance the effector T cells IL-2 and/or IL-15 were added to the co-cultures. First, the effect of both cytokines on the CLEC12A/CD3 bispecific antibody mode of action was assessed in a HL-60 cytotoxicity assay. In this assay, HL-60 cells and healthy donor derived T cells were cultured at an E:T ratio of 5:1. To study the effect of the cytokines in this assay 100 U/mL IL-2 and 5 ng/mL IL-15 was added in the presence of the anti-tetanus toxoid (TT) antibody with wild-type Fc, a monospecific anti-CD3 antibody, a bispecific control antibody TT/CD3 with a modified Fc and a CLEC12A/CD3 bispecific antibody. The results, shown in FIG. 1, show that the combination of IL-2 and IL-15 induced significant background lysis, as observed in the control TT/CD3 bispecific antibody, formatted with same Fc portion as CLEC12A/CD3 bispecific antibody compared to control samples without IL-2 and IL-15. To analyze the effect of an IL-15 moiety in combination with a CLEC12A/CD3 bispecific antibody four concentrations of IL-15 were tested: 2.5, 5, 10 and 20 ng/mL. The experimental conditions were:
Healthy donor T cells as effector cells
HL-60 as target cells
E:T ratio of 1:10
IgG tested at 1000 ng/mL
Day of analysis: 3 days
Read-out: T cell activation (CD25 and CD69 on CD4 and CD8 T cells).

For the assay, 100 μl of the above cells density was used to provide 20,000 HL-60 cells/well. To each assay well was added 50 μl of antibody dilution in IMDM with 10% HS; 100 ul of 0.2×10⁶/ml of CFSE labeled HL-60 cells in IMDM with 10% HS; 50 ul of 2000 cells/ml of MACS sorted CD3+ T cells in IMDM with 10% HS. IL-15 was added on day on day 1.

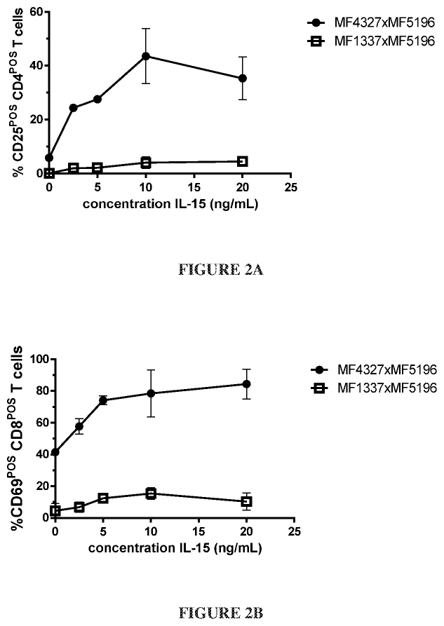

The results show that the activity of the CLEC12A/CD3 bispecific antibody was potentiated by the addition of IL-15. Namely, the enhanced activation of CD4 (FIG. 2A) and CD8 T cells (FIG. 2B) observed correlated with the dose of IL-15. Moreover, the enhanced activity observed in the presence of IL-15 was specific for the CLEC12A/CD3 bispecific antibody and was CLEC12A target specific. No appreciable T cell activation was observed in with the TT/CD3 bispecific control antibody in the presence of IL-15 (PB9124p01).

Accordingly, the combination of IL-15 with the CLEC12A/CD3 bispecific antibody effectively enhanced CLEC12A-target mediated T cell activation.

### SEQUENCE LISTING SUMMARY

| SEQ | | |
|---|---|---|
| ID NO | | |
| 1 | Human | MWIDFFTYSSMSEEVTYADLQFQNSSEMEKIPEIGKFGEKAPPAPSHVW |
| | CLEC12A | RPAALFLTLLCLLLLIGLGVLASMFHVTLKIEMKKMNKLQNISEELQRN |
| | | ISLQLMSNMNISNKIRNLSTTLQTIATKLCRELYSKEQEHKCKPCPRRW |
| | | IWHKDSCYFLSDDVQTWQESKMACAAQNASLLKINNKNALEFIKSQSRS |
| | | YDYWLGLSPEEDSTRGMRVDNIINSSAWVIRNAPDLNNMYCGYINRLYV |
| | | QYYHCTYKKRMICEKMANPVQLGSTYFREA |
| 2 | Human CD3γ | MEQGKGLAVL ILAIILLQGT LAQSIKGNHL VKVYDYQEDG |
| | | SVLLTCDAEA |
| | | KNITWFKDGK MIGFLTEDKK KWNLGSNAKD PRGMYQCKGS |
| | | QNKSKPLQVY |
| | | YRMCQNCIEL NAATISGFLF AEIVSIFVLA VGVYFIAGQD |
| | | GVRQSRASDK |
| | | QTLLPNDQLY QPLKDREDDQ YSHLQGNQLR RN |
| 3 | Human CD3δ | MEHSTFLSGL VLATLLSQVS PFKIPIEELE DRVFVNCNTS |
| | | ITWVEGTVGT |
| | | LLSDITRLDL GKRILDPRGI YRCNGTDIYK DKESTVQVHY |
| | | RMCQSCVELD |
| | | PATVAGIIVT DVIATLLLAL GVFCFAGHET GRLSGAADTQ |
| | | ALLRNDQVYQ |
| | | PLRDRDDAQY SHLGGNWARN K |
| 4 | Human CD3ε | MQSGTHWRVL GLCLLSVGVW GQDGNEEMGG ITQTPYKVSI |
| | | SGTTVILTCP |
| | | QYPGSEILWQ HNDKNIGGDE DDKNIGSDED HLSLKEFSEL |
| | | EQSGYYVCYP |
| | | RGSKPEDANF YLYLRARVCE NCMEMDVMSV ATIVIVDICI |
| | | TGGLLLLVYY |
| | | WSKNRKAKAK PVTRGAGAGG RQRGQNKERP PPVPNPDYEP |
| | | IRKGQRDLYS |
| | | GLNQRRI |
| 5 | Human CD3ζ | MKWKALFTAA ILQAQLPITE AQSFGLLDPK LCYLLDGILF |
| | | IYGVILTALF |
| | | LRVKFSRSAD APAYQQGQNQ LYNELNLGRR EEYDVLDKRR |
| | | GRDPEMGGKP |
| | | QRRKNPQEGL YNELQKDKMA EAYSEIGMKG ERRRGKGHDG |
| | | LYQGLSTATK |
| | | DTYDALHMQA LPPR |
| 6 | Human IL-15 | MRISKPHLRSISIQCYLCLLLNSHFLTEAGIHVFILGCFSAGLPKTEAN |
| | Signal | WVNVISDLKKIEDLIQSMHIDATLYTESDVHPSCKVTAMKCFLLELQVI |
| | peptide | SLESGDASIHDTVENLIILANNSLSSNGNVTESGCKECEELEEKNIKEF |
| | underlined | LQSFVHIVQMFINTS |
| 7 | Human IL- | MAPRRARGCR TLGLPALLLL LLLRPPATRG ITCPPPMSVE |
| | 15Ra | HADIWVKSYS LYSRERYICN SGFKRKAGTS SLTECVLNKA |
| | Signal | TNVAHWTTPS LKCIRDPALV HQRPAPPSTV TTAGVTPQPE |
| | peptide | SLSPSGKEPA ASSPSSNNTA ATTAAIVPGS QLMPSKSPST |
| | underlined | GTTEISSHES SHGTPSQTTA KNWELTASAS HQPPGVYPQG |
| | | HSDTTVAIST STVLLCGLSA VSLLACYLKS RQTPPLASVE |
| | | MEAMEALPVT WGTSSRDEDL ENCSHHL |
| 8 | Soluble | MAPRRARGCR TLGLPALLLL LLLRPPATRG ITCPPPMSVE |
| | human | HADIWVKSYS LYSRERYICN SGFKRKAGTS SLTECVLNKA |
| | IL-15Ra | TNVAHWTTPS LKCIRDPALV HQRPAPPSTV TTAGVTPQPE |
| | Signal | SLSPSGKEPA ASSPSSNNTA ATTAAIVPGS QLMPSKSPST |
| | peptide | GTTEISSHES SHGTPSQTTA KNWELTASAS HQPPGVYPQG |
| | underlined | |
| 9 | CLEC12A | SGYTFTGY |
| | 4327 HCDR1 | |
| 10 | CLEC12A | IINPSGGS |
| | 4327 HCDR2 | |
| 11 | CLEC12A | GTTGDWFDY |
| | 4327 HCDR3 | |
| 12 | 4327 VH | QVQLVQSGAEVKKPGASVKVSCKASGYTFTSYYMHWVRQAPGQGLEWMG |
| | | IINPSGGSTSYAQKFQGRVTMTRDTSTSTVYMELSSLRSEDTAVYYCAK |
| | | GTTGDWFDYWGQGTLVTVSS |
| 13 | CLEC12A | SGYTFTSY |
| | 4331 HCDR1 | |
| 14 | CLEC12A | IINPSGGS |
| | 4331 HCDR2 | |
| 15 | CLEC12A | GNYGDEFDY |
| | 4331 HCDR3 | |
| 16 | 4331 VH | EVQLVQSGAEVKKPGASVKVSCKASGYTFTSYYMHWVRQAPGQGLEWMG |
| | | IINPSGGSTSYAQKFQGRVTMTRDTSTSTVYMELSSLRSEDTAVYYCAR |
| | | GNYGDEFDYWGQGTLVTVSS |
| 17 | CLEC12A | SGYTFTGY |
| | HCDR1 | |
| 18 | CLEC12A | WINPNSGG |
| | HCDR2 | |
| 19 | CLEC12A | DGYFADAFDY |
| | HCDR3 | |
| 20 | CLEC12A VH | QVQLVQSGAEVKKPGASVKVSCKASGYTFTGYYMHWVRQAPGQGLEWMG |
| | | WINPNSGGTNYAQKFQGRVTMTRDTSISTAYMELSRLRSDDTAVYYCAR |
| | | DGYFADAFDYWGQGTLVTVSS |
| 21 | 3056 HCDR1 | GFTFSSYG |
| 22 | 3056 HCDR2 | IWYNGRKQ |
| 23 | 3056 HCDR3 | GTGYNWFDP |
| 24 | MF3056 VH | QVQLVQSGGGVVQPGRSLRLSCVASGFTFSSYGMHWVRQAPGKGLEWVA |
| | | AIWYNGRKQDYADSVKGRFTISRDNSKNTLYLQMNSLRAEDTAVYYCTR |
| | | GTGYNWFDPWGQGTLVTVSS |
| 25 | MF3874 | QVQLVESGGGVVQPGRSLRLSCVASGFTFSSYGMHWVRQAPGKGLEWVA |
| | | AIWYNGRKQDYADSVKGRFTISRDNSKNTLYLQMNSLRAEDTAVYYCTR |
| | | GTGYNWFDPWGQGTLVTVSS |
| 26 | MF3878 | QVQLVQSGGGVVQPGRSLRLSCAASGFTFSSYGMHWVRQAPGKGLEWVA |
| | | AIWYNGRKQDYADSVKGRFTISRDNSKNTLYLQMNSLRAEDTAVYYCTR |
| | | GTGYNWFDPWGQGTLVTVSS |
| 27 | MF3883 | QVQLVQSGGGVVQPGRSLRLSCVASGFTFSSYGMHWVRQAPGKGLEWVA |
| | | VIWYNGRKQDYADSVKGRFTISRDNSKNTLYLQMNSLRAEDTAVYYCTR |
| | | GTGYNWFDPWGQGTLVTVSS |
| 28 | MF3886 | QVQLVQSGGGVVQPGRSLRLSCVASGFTFSSYGMHWVRQAPGKGLEWVA |
| | | AIWYNGRKQDYADSVKGRFTISRDNSKNTLYLQMNSLRAEDTAVYYCAR |
| | | GTGYNWFDPWGQGTLVTVSS |
| 29 | MF3891 | QVQLVESGGGVVQPGRSLRLSCAASGFTFSSYGMHWVRQAPGKGLEWVA |
| | | AIWYNGRKQDYADSVKGRFTISRDNSKNTLYLQMNSLRAEDTAVYYCAR |
| | | GTGYNWFDPWGQGTLVTVSS |
| 30 | 3896 HCDR2 | IWYSGSKKN |
| 31 | 3896 VH | QVQLVESGGGVVQPGRSLRLSCAASGFTFRSYGMHWVRQAPGKGLEWVA |
| | | IIWYSGSKKNYADSVKGRFTISRDNSKNTLYLQMNSLRAEDTAVYYCAR |
| | | GTGYNWFDPWGQGTLVTVSS |
| 32 | MF5192 | IWYHGRKQ |
| | HCDR2 | |
| 33 | MF5192 | QVQLVQSGGGVVQPGRSLRLSCVASGFTFSSYGMHWVRQAPGKGLEWVA |
| | | AIWYHGRKQDYADSVKGRFTISRDNSKNTLYLQMNSLRAEDTAVYYCTR |
| | | GTGYNWFDPWGQGTLVTVSS |
| 34 | MF5193 | IWYHARKQ |
| | HCDR2 | |
| 35 | MF5193 | QVQLVQSGGGVVQPGRSLRLSCVASGFTFSSYGMHWVRQAPGKGLEWVA |
| | | AIWYHARKQDYADSVKGRFTISRDNSKNTLYLQMNSLRAEDTAVYYCTR |
| | | GTGYNWFDPWGQGTLVTVSS |
| 36 | MF5196 | IWYNARKQ |
| | HCDR2 | |
| 37 | MF5196 | QVQLVQSGGGVVQPGRSLRLSCVASGFTFSSYGMHWVRQAPGKGLEWVA |
| | | AIWYNARKQDYADSVKGRFTISRDNSKNTLYLQMNSLRAEDTAVYYCTR |
| | | GTGYNWFDPWGQGTLVTVSS |
| 38 | MF5603 | QVQLVQSGGGVVQPGRSLRLSCVASGFTFSSYGMHWVRQAPGKGLEWVA |
| | | AIWYNARKQEYIDSVKGRFTISRDNSKNTLYLQMNSLRAEDTAVYYCTR |
| | | GTGYNWFDPWGQGTLVTVSS |
| 39 | MF5616 | QVQLVQSGGGVVQPGRSLRLSCVASGFTFSSYGMHWVRQAPGKGLEWVA |
| | | AIWYNARKQEYNDSVKGRFTISRDNSKNTLYLQMNSLRAEDTAVYYCTR |
| | | GTGYNWFDPWGQGTLVTVSS |
| 40 | MF5626 | QVQLVQSGGGVVQPGRSLRLSCVASGFTFSSYGMHWVRQAPGKGLEWVA |
| | | AIWYNARKQEYRDSVKGRFTISRDNSKNTLYLQMNSLRAEDTAVYYCTR |
| | | GTGYNWFDPWGQGTLVTVSS |
| 41 | MF5630 | QVQLVQSGGGVVQPGRSLRLSCVASGFTFSSYGMHWVRQAPGKGLEWVA |
| | | AIWYNARKQDYADSVKGRFTISRDNSKNTLYLQMNSLRAEDTAVYYCTR |
| | | GTGYNWYDPWGQGTLVTVSS |
| 42 | MF5648 | QVQLVQSGGGVVQPGRSLRLSCVASGFTFSSYGMHWVRQAPGKGLEWVA |
| | | AIWYNARKQEYLDSVKGRFTISRDNSKNTLYLQMNSLRAEDTAVYYCTR |
| | | GTGYNWFDPWGQGTLVTVSS |
| 43 | MF5661 | QVQLVQSGGGVVQPGRSLRLSCVASGFTFSSYGMHWVRQAPGKGLEWVA |
| | | QIWYNARKQEYSDSVKGRFTISRDNSKNTLYLQMNSLRAEDTAVYYCTR |
| | | GTGYNWFDPWGQGTLVTVSS |
| 44 | MF5694 | QVQLVQSGGGVVQPGRSLRLSCVASGFTFSSYGMHWVRQAPGKGLEWVA |
| | | AIWYNARKQEYSDSVKGRFTISRDNSKNTLYLQMNSLRAEDTAVYYCTR |
| | | GTGYNWFDPWGQGTLVTVSS |
| 45 | MF5197 | IWYNTRKQ |
| | HCDR2 | |
| 46 | MF5197 | QVQLVQSGGGVVQPGRSLRLSCVASGFTFSSYGMHWVRQAPGKGLEWVA |
| | | AIWYNTRKQDYADSVKGRFTISRDNSKNTLYLQMNSLRAEDTAVYYCTR |
| | | GTGYNWFDPWGQGTLVTVSS |
| 47 | MF5351 | IWYDGKNT |
| | HCDR2 | |
| 48 | MF5351 | QVQLVQSGGGVVQPGRSLRLSCAASGFTFSSYGMHWVRQAPGKGLEWVA |
| | | MIWYDGKNTYYADSVKGRFTISRDNSKNTLYLQMNSLRAEDTAVYYCTR |
| | | GTGYNWFDPWGQGTLVTVSS |
| 49 | MF5354 | IYYDGSRT |
| | HCDR2 | |
| 50 | MF5354 | QVQLVQSGGGVVQPGRSLRLSCAASGFTFSGYGMHWVRQAPGKGLEWVA |
| | | QIYYDGSRTYYADSVKGRFTISRDNSKNTLYLQMNSLRAEDTAVYYCTR |
| | | GTGYNWFDPWGQGTLVTVSS |
| 51 | MF5356 | IWHDGRKT |
| | HCDR2 | |
| 52 | MF5356 | QVQLVESGGGVVQPGRSLRLSCAASGFTFSKYGMHWVRQAPGKGLEWVA |
| | | QIWHDGRKTYYADSVKGRFTISRDNSKNTLYLQMNSLRAEDTAVYYCTR |
| | | GTGYNWFDPWGQGTLVTVSS |
| 53 | VL CDR1 | QSISSY |
| 54 | VL CDR2 | AASSLQS |
| 55 | VL CDR3 | QQSYSTP |
| 56 | IgVk1- | DIQMTQSPSSLSASVGDRVTITCRASQSISSYLNWYQQKPGKAPKLLIY |
| | 39*01 | AASSLQSGVPSRFSGSGSGTDFTLTISSLQPEDFATYYCQQSYSTP |
| 57 | Common | DIQMTQSPSSLSASVGDRVTITCRASQSISSYLNWYQQKPGKAPKLLIY |
| | Light | AASSLQSGVPSRFSGSGSGTDFTLTISSLQPEDFATYYCQQ |
| | Chain | SYSTPPTFGQGTKVEIK |
| | IgKV1*39/ | |
| | jk1 | |
| 58 | Common | DIQMTQSPSSLSASVGDRVTITCRASQSISSYLNWYQQKPGKAPK |
| | Light | LLIYAASSLQSGVPSRFSGSGSGTDFTLTISSLQPEDFATYYCQQ |
| | Chain | SYSTPPITFGQGTRLEIK |
| | IgKV1*39/ | |
| | jk5 | |
| 59 | MF1337 TT | EVQLVETGAEVKKPGASVKVSCKASDYIFTKYDINWVRQAPGQGLEWMG |
| | VH | WMSANTGNTGYAQKFQGRVTMTRDTSINTAYMELSSLTSGDTAVYFCAR |
| | | SSLFKTETAPYYHFALDVWGQGTTVTVSS |
| 60 | 3056 HCDR1 | SYGMH |
| 61 | MF1207 TT | EVQLVQSGAEVKKPGASVKVSCKASGYTFTSYYMHWVRQAPGQGLEWMG |
| | VH | IINPSGGSTSYAQKFQGRVTMTRDTSTSTVYMELSSLRSEDTAVYYCAR |
| | | MWRLNPVGMDYWGQGTLVTVSS |

1. A method of activating a T cell in a subject, the method comprising administering to the subject a CLEC12A/CD3 bispecific antibody and an IL-15 moiety.

2. The method of claim 1, wherein administering the CLEC12A/CD3 bispecific antibody and the IL-5 moiety activates the T cell to specifically target a CLEC12A expressing cell.

3. The method of claim 1, wherein administering the CLEC12A/CD3 bispecific antibody and the IL-5 moiety activates the T cell to specifically target and lyse a CLEC12A expressing cell.

4. The method of claim 1, wherein the subject has a cancer.

5. A method of treating a cancer in a subject, the method comprising administering to the subject a CLEC12A/CD3 bispecific antibody and an IL-15 moiety.

6. The method of any one of the preceding claims, wherein the subject is human.

7. The method of any one of claims 4 to 6, wherein the cancer is of myeloid origin.

8. The method of any one of claims 4 to 7, wherein the cancer is selected from leukemia or pre-leukemia.

9. The method of any one of claims 4 to 8, wherein the cancer is selected from acute myeloid leukemia (AML), myelodysplastic syndrome (MDS), and chronic myelogenous leukemia (CML).

10. The method of any one of claims 1 to 9, wherein the CLEC12A/CD3 bispecific antibody and the IL-15 moiety are administered to the subject concurrently.

11. The method of any one of claims 1 to 9, wherein the CLEC12A/CD3 bispecific antibody is administered to the subject prior to the IL-15 moiety.

12. The method of any one of claims 1 to 9, wherein the IL-15 moiety is administered to the subject prior to the CLEC12A/CD3 bispecific antibody.

13. The method of any one of the preceding claims, wherein the binding affinity of the CLEC12A/CD3 bispecific antibody for CLEC12A on tumor cells is at least 2 times, 4 times 6 times, 10 times, 20 times, 30 times, 40 times or 50 times higher than the affinity of binding to CD3.

14. The method of any one of the preceding claims, wherein the CLEC12A/CD3 bispecific antibody binds CD3ε.

15. The method of any one of the preceding claims, wherein the CLEC12A/CD3 bispecific antibody comprises a first heavy chain variable region that binds human CLEC12A, wherein the first heavy chain variable region comprises:
(a) a heavy chain CDR1 comprising the amino acid sequence SGYTFTGY (SEQ ID NO: 9), a heavy chain CDR2 comprising the amino acid sequence IINPSGGS (SEQ ID NO: 10), and a heavy chain CDR3 comprising the amino acid sequence GTTGDWFDY (SEQ ID NO: 11);
(b) a heavy chain CDR1 comprising the amino acid sequence SGYTFTSY (SEQ ID NO: 13), a heavy chain CDR2 comprising the amino acid sequence IINPSGGS (SEQ ID NO: 14), and a heavy chain CDR3 comprising the amino acid sequence GNYGDEFDY (SEQ ID NO: 15); or
(c) a heavy chain CDR1 comprises the amino acid sequence SGYTFTGY (SEQ ID NO: 17), a heavy chain CDR2 comprising the amino acid sequence WINPNSGG (SEQ ID NO: 18), and a heavy chain CDR3 comprising the amino acid sequence DGYFADAFDY (SEQ ID NO: 19).

16. The method of any one of the preceding claims, wherein the CLEC12A/CD3 bispecific antibody comprises a first heavy chain variable region that binds human CLEC12A and wherein the first heavy chain variable region comprises the HCDR1, HCDR2 and HCDR3 of the VH set forth in SEQ ID NOs: 12, 16 or 20.

17. The method of any one of the preceding claims, wherein the CLEC12A/CD3 bispecific antibody comprises a first heavy chain variable region that binds human CLEC12A and wherein the first heavy chain variable region comprises an amino acid sequence at least 90% identical to the amino acid sequence set forth in SEQ ID NOs: 12, 16 or 20.

18. The method of any one of the preceding claims, wherein the CLEC12A/CD3 bispecific antibody comprises a first heavy chain variable region that binds human CLEC12A, wherein the first heavy chain variable region comprises the amino acid sequence set forth in SEQ ID NOs: 12, 16 or 20.

19. The method of any one of the preceding claims, wherein the CLEC12A/CD3 bispecific antibody comprises a second heavy chain variable region that binds CD3, wherein the second heavy chain variable region comprises:
(a) a heavy chain CDR1 comprising the amino acid sequence GFTFSSYG (SEQ ID NO: 21), a heavy chain CDR2 sequence comprising the amino acid sequence IWYNGRKQ (SEQ ID NO: 22), and a heavy chain CDR3 comprising the amino acid sequence GTGYNWFDP (SEQ ID NO: 23);
(b) a heavy chain CDR1 comprising the amino acid sequence GFTFSSYG (SEQ ID NO: 21), a heavy chain CDR2 sequence comprising the amino acid sequence IWYSGSKKN(SEQ ID NO: 30), and a heavy chain CDR3 comprising the amino acid sequence GTGYNWFDP (SEQ ID NO: 23);
(c) a heavy chain CDR1 comprising the amino acid sequence GFTFSSYG (SEQ ID NO: 21), a heavy chain CDR2 sequence comprising the amino acid sequence IWYHGRKQ (SEQ ID NO: 32), and a heavy chain CDR3 comprising the amino acid sequence GTGYNWFDP (SEQ ID NO: 23);
(d) a heavy chain CDR1 comprising the amino acid sequence GFTFSSYG (SEQ ID NO: 21), a heavy chain CDR2 sequence comprising the amino acid sequence IWYHARKQ (SEQ ID NO: 34), and a heavy chain CDR3 comprising the amino acid sequence GTGYNWFDP (SEQ ID NO: 23);
(e) a heavy chain CDR1 comprising the amino acid sequence GFTFSSYG (SEQ ID NO: 21), a heavy chain CDR2 sequence comprising the amino acid sequence IWYNARKQ (SEQ ID NO: 36), and a heavy chain CDR3 comprising the amino acid sequence GTGYNWFDP (SEQ ID NO: 23);
(f) a heavy chain CDR1 comprising the amino acid sequence GFTFSSYG (SEQ ID NO: 21), a heavy chain CDR2 sequence comprising the amino acid sequence IWYNTRKQ (SEQ ID NO: 45), and a heavy chain CDR3 comprising the amino acid sequence GTGYNWFDP (SEQ ID NO: 23);
(g) a heavy chain CDR1 comprising the amino acid sequence GFTFSSYG (SEQ ID NO: 21), a heavy chain CDR2 sequence comprising the amino acid sequence IWYDGKNT (SEQ ID NO: 47), and a heavy chain CDR3 comprising the amino acid sequence GTGYNWFDP (SEQ ID NO: 23);
(h) a heavy chain CDR1 comprising the amino acid sequence GFTFSGYG (SEQ ID NO: 21), a heavy chain CDR2 sequence comprising the amino acid sequence IYYDGSRT (SEQ ID NO: 49), and a heavy chain CDR3 comprising the amino acid sequence GTGYNWFDP (SEQ ID NO: 23); or
(i) a heavy chain CDR1 comprising the amino acid sequence GFTFSKYG (SEQ ID NO: 21), a heavy chain CDR2 sequence comprising the amino acid sequence IWHDGRKT (SEQ ID NO: 51), and a heavy chain CDR3 comprising the amino acid sequence GTGYNWFDP (SEQ ID NO: 23).

20. The method of any one of the preceding claims, wherein the CLEC12A/CD3 bispecific antibody comprises a second heavy chain variable region that binds human CD3, wherein the second heavy chain variable region comprises the HCDR1, HCDR2 and HCDR3 of the VH selected from the group forth in SEQ ID NOs: 24-29, 31, 33, 35, 37-44, 46, 48, 50 and 52.

21. The method of any one of the preceding claims, wherein the CLEC12A/CD3 bispecific antibody comprises a second heavy chain variable region that binds human CD3, wherein the second heavy chain variable region comprises an amino acid sequence at least 90% identical to the amino acid sequence set forth in SEQ ID NOs: 24-29, 31, 33, 35, 37-44, 46, 48, 50 or 52.

22. The method of any one of the preceding claims, wherein the CLEC12A/CD3 bispecific antibody comprises a second heavy chain variable region that binds human CD3 and wherein the second heavy chain variable region comprises an amino acid sequence selected from the group set forth in SEQ ID NOs: 24-29, 31, 33, 35, 37-44, 46, 48, 50 and 52.

23. The method of any one of the preceding claims, wherein the CLEC12A/CD3 bispecific antibody comprises a first heavy chain variable region that binds human CLEC12A, wherein the amino acid sequence of the first VH is selected from SEQ ID NOs: 12, 16 and 20; and a second heavy chain variable region that binds human CD3, wherein the amino acid sequence of the second VH is selected from SEQ ID NOs: 24-29, 31, 33, 35, 37-44, 46, 48, 50 or 52.

24. The method of any one of the preceding claims, wherein the CLEC12A/CD3 bispecific antibody comprises a first heavy chain variable region that binds human CLEC12A, wherein the first VH comprises a heavy chain CDR1 comprising the amino acid sequence SGYTFTGY (SEQ ID NO: 9), a heavy chain CDR2 comprising the amino acid sequence IINPSGGS (SEQ ID NO: 10), and a heavy chain CDR3 comprising the amino acid sequence GTTGDWFDY (SEQ ID NO: 11); and a second heavy chain variable region that binds human CD3, wherein the second VH comprises a heavy chain CDR1 comprising the amino acid sequence GFTFSSYG (SEQ ID NO: 21), a heavy chain CDR2 comprising the amino acid sequence IWYNARKQ (SEQ ID NO: 36), and a heavy chain CDR3 comprising the amino acid sequence GTGYNWFDP (SEQ ID NO: 23).

25. The method of any one of the preceding claims, wherein the CLEC12A/CD3 bispecific antibody comprises a first heavy chain variable region comprising the amino acid sequence set forth in SEQ ID NO: 12, and a second heavy chain variable region comprising the amino acid sequence set forth in SEQ ID NO: 37.

26. The method of any one of the preceding claims, wherein the CLEC12A/CD3 bispecific antibody comprises a first VH/VL binding region that binds human CLEC12A, and a second VH/VL region that binds human CD3, wherein the VL of the first and second VH/VL binding regions comprises a common light chain.

27. The method of claim 26, wherein the common light chain comprises a variable light chain CDR1 comprising the amino acid sequence QSISSY (SEQ ID NO: 53), a light chain CDR2 comprising the amino acid sequence AASSLQS (SEQ ID NO: 54), and a light chain CDR3 comprising the amino acid sequence QQSYSTP (SEQ ID NO: 55).

28. The method of claim 26 or 27, wherein the first and second VL each comprise a variable light chain that is at least 90% identical to the amino acid sequence set forth in SEQ ID NO: 57 or SEQ ID NO: 58.

29. The method of any one of the preceding claims, wherein the constant region of the heavy chain comprising the CLEC12A binding variable region and the constant region of the heavy chain comprising the CD3 binding variable region of the CLEC12A/CD3 bispecific antibody comprise compatible heterodimerization domains.

30. The method of any one of the preceding claims, wherein the CLEC12A/CD3 bispecific antibody is an IgG antibody which comprises an Fc portion comprising a mutated CH2 and/or lower hinge domain, wherein binding of said Fc portion with human Fc-gamma receptors is reduced.

31. The method of claim 30, wherein the mutant CH2 and/or lower hinge domain comprises an amino substitution at position 235 and/or 236 (EU numbering).

32. The method of claim 31, wherein the mutant CH2 and/or lower hinge domain comprises an L235G and/or G236R substitution.

33. The method of any one of the preceding claims, wherein the IL-15 moiety is a naturally-occurring IL-15, recombinant IL-15, synthetic IL-15, modified IL-15, PEGylated IL-15, a fusion protein comprising IL-15 and a heterologous fusion partner, an IL-15 mimetic or a functional fragment of any one thereof.

34. The method of any one of the preceding claims, wherein the IL-15 moiety is human IL-15.

35. The method of claim 34, wherein the human IL-15 is recombinant human IL-15 (rhIL-15).

36. The method of claim 33, wherein the IL-15 moiety is a complex comprising IL-15 and soluble IL-15Ra (sIL-15 Ra).

37. The method of claim 33, wherein the IL-15 moiety is IL-15RaSu/Fc.

38. The method of claim 33, wherein the IL-15 moiety is a conjugate of IL-15 and a water soluble polymer.

39. A pharmaceutical composition comprising a CLEC12A/CD3 bispecific antibody and an IL-15 moiety.

40. The pharmaceutical composition of claim 39, wherein the CLEC12A/CD3 bispecific antibody and the IL-15 moiety are provided in a single formulation.

41. The pharmaceutical composition of claim 39, wherein the CLEC12A/CD3 bispecific antibody and the IL-15 moiety are provided in separate formulations.

42. A kit comprising a CLEC12A/CD3 bispecific antibody, an IL-15 moiety and instructions for using the CLEC12A/CD3 bispecific antibody and the IL-15 moiety in the method of any one of claims 1-38.

43. A CLEC12A/CD3 bispecific antibody for use in activating a T cell in a subject, wherein the CLEC12A/CD3 bispecific antibody is administered simultaneously, separately or sequentially with an IL-15 moiety.

44. A CLEC12A/CD3 bispecific antibody for use in the manufacture of a medicament for activating a T cell in a subject, wherein the CLEC12A/CD3 bispecific antibody is administered simultaneously, separately or sequentially with an IL-15 moiety.

45. A product comprising a CLEC12A/CD3 bispecific antibody and an IL-15 moiety as a combined preparation for simultaneous, separate or sequential use in activating a T cell in a subject.

46. A CLEC12A/CD3 bispecific antibody and an IL-15 moiety for use in the treatment of a cancer in a subject.


SEQUENCE LISTING (63 sequences)

SEQ ID NO: 1 - protein, 275 aa, Homo sapiens
MWIDFFTYSSMSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 2 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 3 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 8 - protein, 200 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 9 - protein, 8 aa, Artificial Sequence
  CLEC12A 4327 HCDR1
SGYTFTGY

SEQ ID NO: 10 - protein, 8 aa, Artificial Sequence
  CLEC12A 4327 HCDR2
IINPSGGS

SEQ ID NO: 11 - protein, 9 aa, Artificial Sequence
  CLEC12A 4327 HCDR3
GTTGDWFDY

SEQ ID NO: 12 - protein, 118 aa, Artificial Sequence
  4327 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAKGTTGDWFDYWGQGTLVTVSS

SEQ ID NO: 13 - protein, 8 aa, Artificial Sequence
  CLEC12A 4331 HCDR1
SGYTFTSY

SEQ ID NO: 14 - protein, 8 aa, Artificial Sequence
  CLEC12A 4331 HCDR2
IINPSGGS

SEQ ID NO: 15 - protein, 9 aa, Artificial Sequence
  CLEC12A 4331 HCDR3
GNYGDEFDY

SEQ ID NO: 16 - protein, 118 aa, Artificial Sequence
  4331 VH
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGNYGDEFDYWGQGTLVTVSS

SEQ ID NO: 17 - protein, 8 aa, Artificial Sequence
  CLEC12A HCDR1
SGYTFTGY

SEQ ID NO: 18 - protein, 8 aa, Artificial Sequence
  CLEC12A HCDR2
WINPNSGG

SEQ ID NO: 19 - protein, 10 aa, Artificial Sequence
  CLEC12A HCDR3
DGYFADAFDY

SEQ ID NO: 20 - protein, 119 aa, Artificial Sequence
  CLEC12A VH
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGYFADAFDYWGQGTLVTVSS

SEQ ID NO: 21 - protein, 8 aa, Artificial Sequence
  3056 HCDR1
GFTFSSYG

SEQ ID NO: 22 - protein, 8 aa, Artificial Sequence
  3056 HCDR2
IWYNGRKQ

SEQ ID NO: 23 - protein, 9 aa, Artificial Sequence
  3056 HCDR3
GTGYNWFDP

SEQ ID NO: 24 - protein, 118 aa, Artificial Sequence
  MF3056 VH
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 25 - protein, 118 aa, Artificial Sequence
  MF3874
QVQLVESGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 26 - protein, 118 aa, Artificial Sequence
  MF3878
QVQLVQSGGGVVQPGRSLRLSCAASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 27 - protein, 118 aa, Artificial Sequence
  MF3883
QVQLVQSGGGVVQPGRSLRLSCVASGFTFSSYGMHWVRQAPGKGLEWVAVIWYNGRKQDYADSVKGRFTISRDNSKNTLYLQMNSLRAEDTAVYYCTRGTGYNWFDPWGQGTLVTVSS

SEQ ID NO: 28 - protein, 118 aa, Artificial Sequence
  MF3886
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCARGTGYNWFDPWGQGTLVTVSS

SEQ ID NO: 29 - protein, 118 aa, Artificial Sequence
  MF3891
QVQLVESGGGVVQPGRSLRLSCAASGFTFSSYGMHWVRQAPGKGLEWVAAIWYNGRKQDYADSVKGRFTISRDNSKNTLYLQMNSLRAEDTAVYYCARGTGYNWFDPWGQGTLVTVSS

SEQ ID NO: 30 - protein, 9 aa, Artificial Sequence
  3896 HCDR2
IWYSGSKKN

SEQ ID NO: 31 - protein, 118 aa, Artificial Sequence
  3896 VH
QVQLVESGGGVVQPGRSLRLSCAASGFTFRSYGMHWVRQAPGKGLEWVAIIWYSGSKKNYADSVKGRFTISRDNSKNTLYLQMNSLRAEDTAVYYCARGTGYNWFDPWGQGTLVTVSS

SEQ ID NO: 32 - protein, 8 aa, Artificial Sequence
  MF5192 HCDR2
IWYHGRKQ

SEQ ID NO: 33 - protein, 118 aa, Artificial Sequence
  MF5192
QVQLVQSGGGVVQPGRSLRLSCVASGFTFSSYGMHWVRQAPGKGLEWVAAIWYHGRKQDYADSVKGRFTISRDNSKNTLYLQMNSLRAEDTAVYYCTRGTGYNWFDPWGQGTLVTVSS

SEQ ID NO: 34 - protein, 8 aa, Artificial Sequence
  MF5193 HCDR2
IWYHARKQ

SEQ ID NO: 35 - protein, 118 aa, Artificial Sequence
  MF5193
QVQLVQSGGGVVQPGRSLRLSCVASGFTFSSYGMHWVRQAPGKGLEWVAAIWYHARKQDYADSVKGRFTISRDNSKNTLYLQMNSLRAEDTAVYYCTRGTGYNWFDPWGQGTLVTVSS

SEQ ID NO: 36 - protein, 8 aa, Artificial Sequence
  MF5196 HCDR2
IWYNARKQ

SEQ ID NO: 37 - protein, 118 aa, Artificial Sequence
  MF5196
QVQLVQSGGGVVQPGRSLRLSCVASGFTFSSYGMHWVRQAPGKGLEWVAAIWYNARKQDYADSVKGRFTISRDNSKNTLYLQMNSLRAEDTAVYYCTRGTGYNWFDPWGQGTLVTVSS

SEQ ID NO: 38 - protein, 118 aa, Artificial Sequence
  MF5603
QVQLVQSGGGVVQPGRSLRLSCVASGFTFSSYGMHWVRQAPGKGLEWVAAIWYNARKQEYIDSVKGRFTISRDNSKNTLYLQMNSLRAEDTAVYYCTRGTGYNWFDPWGQGTLVTVSS

SEQ ID NO: 39 - protein, 118 aa, Artificial Sequence
  MF5616
QVQLVQSGGGVVQPGRSLRLSCVASGFTFSSYGMHWVRQAPGKGLEWVAAIWYNARKQEYNDSVKGRFTISRDNSKNTLYLQMNSLRAEDTAVYYCTRGTGYNWFDPWGQGTLVTVSS

SEQ ID NO: 40 - protein, 118 aa, Artificial Sequence
  MF5626
QVQLVQSGGGVVQPGRSLRLSCVASGFTFSSYGMHWVRQAPGKGLEWVAAIWYNARKQEYRDSVKGRFTISRDNSKNTLYLQMNSLRAEDTAVYYCTRGTGYNWFDPWGQGTLVTVSS

SEQ ID NO: 41 - protein, 118 aa, Artificial Sequence
  MF5630
QVQLVQSGGGVVQPGRSLRLSCVASGFTFSSYGMHWVRQAPGKGLEWVAAIWYNARKQDYADSVKGRFTISRDNSKNTLYLQMNSLRAEDTAVYYCTRGTGYNWYDPWGQGTLVTVSS

SEQ ID NO: 42 - protein, 118 aa, Artificial Sequence
  MF5648
QVQLVQSGGGVVQPGRSLRLSCVASGFTFSSYGMHWVRQAPGKGLEWVAAIWYNARKQEYLDSVKGRFTISRDNSKNTLYLQMNSLRAEDTAVYYCTRGTGYNWFDPWGQGTLVTVSS

SEQ ID NO: 43 - protein, 118 aa, Artificial Sequence
  MF5661
QVQLVQSGGGVVQPGRSLRLSCVASGFTFSSYGMHWVRQAPGKGLEWVAQIWYNARKQEYSDSVKGRFTISRDNSKNTLYLQMNSLRAEDTAVYYCTRGTGYNWFDPWGQGTLVTVSS

SEQ ID NO: 44 - protein, 118 aa, Artificial Sequence
  MF5694
QVQLVQSGGGVVQPGRSLRLSCVASGFTFSSYGMHWVRQAPGKGLEWVAAIWYNARKQEYSDSVKGRFTISRDNSKNTLYLQMNSLRAEDTAVYYCTRGTGYNWFDPWGQGTLVTVSS

SEQ ID NO: 45 - protein, 8 aa, Artificial Sequence
  MF5197 HCDR2
IWYNTRKQ

SEQ ID NO: 46 - protein, 118 aa, Artificial Sequence
  MF5197
QVQLVQSGGGVVQPGRSLRLSCVASGFTFSSYGMHWVRQAPGKGLEWVAAIWYNTRKQDYADSVKGRFTISRDNSKNTLYLQMNSLRAEDTAVYYCTRGTGYNWFDPWGQGTLVTVSS

SEQ ID NO: 47 - protein, 8 aa, Artificial Sequence
  MF5351 HCDR2
IWYDGKNT

SEQ ID NO: 48 - protein, 118 aa, Artificial Sequence
  MF5351
QVQLVQSGGGVVQPGRSLRLSCAASGFTFSSYGMHWVRQAPGKGLEWVAMIWYDGKNTYYADSVKGRFTISRDNSKNTLYLQMNSLRAEDTAVYYCTRGTGYNWFDPWGQGTLVTVSS

SEQ ID NO: 49 - protein, 8 aa, Artificial Sequence
  MF5354 HCDR2
IYYDGSRT

SEQ ID NO: 50 - protein, 118 aa, Artificial Sequence
  MF5354
QVQLVQSGGGVVQPGRSLRLSCAASGFTFSGYGMHWVRQAPGKGLEWVAQIYYDGSRTYYADSVKGRFTISRDNSKNTLYLQMNSLRAEDTAVYYCTRGTGYNWFDPWGQGTLVTVSS

SEQ ID NO: 51 - protein, 8 aa, Artificial Sequence
  MF5356 HCDR2
IWHDGRKT

SEQ ID NO: 52 - protein, 118 aa, Artificial Sequence
  MF5356
QVQLVESGGGVVQPGRSLRLSCAASGFTFSKYGMHWVRQAPGKGLEWVAQIWHDGRKTYYADSVKGRFTISRDNSKNTLYLQMNSLRAEDTAVYYCTRGTGYNWFDPWGQGTLVTVSS

SEQ ID NO: 53 - protein, 6 aa, Artificial Sequence
  VL CDR1
QSISSY

SEQ ID NO: 54 - protein, 7 aa, Artificial Sequence
  VL CDR2
AASSLQS

SEQ ID NO: 55 - protein, 7 aa, Artificial Sequence
  VL CDR3
QQSYSTP

SEQ ID NO: 56 - protein, 95 aa, Artificial Sequence
  IgVk1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 57 - protein, 107 aa, Artificial Sequence
  Common Light Chain IgKV1*39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 58 - protein, 108 aa, Artificial Sequence
  Common Light Chain IgKV1*39/jk5
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 59 - protein, 127 aa, Artificial Sequence
  MF1337 TT VH
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 60 - protein, 5 aa, Artificial Sequence
  3056 HCDR1
SYGMH

SEQ ID NO: 61 - protein, 120 aa, Artificial Sequence
  MF1207 TT VH
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARMWRLNPVGMDYWGQGTLVTVSS

SEQ ID NO: 62 - protein, 8 aa, Artificial Sequence
  MF5354 HC CDR1
GFTFSGYG

SEQ ID NO: 63 - protein, 8 aa, Artificial Sequence
  MF5356 HC CDR1
GFTFSKYG